(19) United States
(12) Patent Application Publication
(10) Pub. No.: US 2017/0260285 A1
(43) Pub. Date: Sep. 14, 2017

(54) METHODS FOR TREATING CANCER

(71) Applicant: UNIVERSITY OF IOWA RESEARCH FOUNDATION, Iowa City, IA (US)

(72) Inventor: Andrean Burnett, Iowa City, IA (US)

(73) Assignee: UNIVERSITY OF IOWA RESEARCH FOUNDATION, Iowa City, IA (US)

(21) Appl. No.: 15/529,315

(22) PCT Filed: Nov. 24, 2015
(86) PCT No.: PCT/US15/62510

§ 371 Date: May 24, 2017

Related U.S. Application Data
(60) Provisional application No. 62/083,751, filed on Nov. 24, 2014.
(60) Provisional application No. 62/182,286, filed on Jun. 19, 2015.

(51) Int. Cl. C07K 16/30; C07K 16/22; C07K 16/46; C07K 16/24

(52) U.S. Cl. CPC C07K 16/30; C07K 16/241; C07K 16/245; C07K 16/22; C07K 16/24; C07K 16/468; C07K 2317/73

(57) ABSTRACT

The present invention relates to pharmaceutical compositions and methods of treating cancer in a subject, the method comprising administering to the subject a combination therapy comprising administering (1) at least one anti-cancer therapy, and (2) a pharmaceutical composition comprising a pharmaceutically acceptable carrier and an amount of an IL-1α inhibitor, wherein the combination therapy is effective to reduce at least one symptom of the cancer in the subject.

### RELATED APPLICATIONS

This application claims the benefit of priority of U.S. Provisional Application Ser. No. 62/083,751 filed on Nov. 24, 2014 and U.S. Provisional Application Ser. No. 62/182,286 filed on Jun. 19, 2015, which applications are herein incorporated by reference.

### STATEMENT REGARDING FEDERALLY SPONSORED RESEARCH

This invention was made with government support under R01DE024550 and K01CA134941 awarded by the National Institutes of Health. The government has certain rights in the invention.

### BACKGROUND OF THE INVENTION

Cancer remains one of the leading causes of death and morbidity in developed nations. Head and neck squamous cell carcinoma (HNSCC) develops from the mucosal linings of the upper aerodigestive tract, including the nasal cavity and paranasal sinuses; the nasopharynx, the hypopharynx, larynx, and trachea; and the oral cavity and oropharynx. Squamous cell carcinoma (SCC) is the most frequent malignant tumor of the head and neck region. HNSCC is the sixth leading cancer by incidence worldwide. There are 500,000 new cases a year worldwide, with two-thirds occur in industrialized nations. HNSCC usually develops in males in their 60s and 70s, and is often caused by tobacco and alcohol consumption and infection with high-risk types of human papillomavirus (HPV). SCC often develops from preexisting dysplastic lesions. The five-year survival rate of patients with HNSCC is about 40-50% and has been so for the last few decades or so despite improvements in surgical techniques and development of targeted therapies.

Current treatments for HNSCC include surgery, radiation therapy, chemotherapy and photodynamic therapy. While increasingly successful, each of these treatments still causes numerous undesired side effects. For example, surgery results in pain, traumatic injury to healthy tissue, and scarring. Surgery can be particularly difficult if the cancer is near the larynx and can result in the patient being unable to speak. Chemotherapy in throat cancer is not generally used to cure the cancer as such, but rather to prevent metastases in other parts of the body. Further, radiotherapy and chemotherapy cause nausea, immune suppression, gastric ulceration, and secondary tumorigenesis. Moreover, current treatments have not had much success at improving survival. Early-stage patients have a high risk of developing secondary tumors after local control is achieved. Two years after standard treatment, 50-60% of patients will be diagnosed with local invasion and regional lymph node metastases, and 15-25% will be found with distant metastases. In actuality, the rate of distant metastases is far greater since distant metastases are often very difficult to detect. Autopsies of HNSCC patients have shown distant metastases in up to 50% of HNSCC fatalities. Therefore, the identification and understanding of molecular mechanisms associated with chemo-/radio-therapy efficacy and resistance in addition to the invasive and metastatic properties of HNSCC cells are needed in order to improve patient survival.

Accordingly, a more effective, simple-to-administer, and efficient treatment for cancer is needed.

### SUMMARY OF THE INVENTION

The present invention provides in certain embodiments a method of treating cancer in a subject, the method comprising administering to the subject a combination therapy comprising administering (1) at least one anti-cancer therapy, and (2) a pharmaceutical composition comprising a pharmaceutically acceptable carrier and an amount of an IL-la inhibitor, wherein the combination therapy is effective to reduce at least one symptom of the cancer in the subject.

In certain embodiments, the subject is a mammal. In certain embodiments, the mammal is a human.

The present invention provides in certain embodiments a pharmaceutical composition for treating cancer in a subject comprising a pharmaceutically acceptable carrier, an amount of chemotherapeutic agent, and an amount of an IL-1α inhibitor, wherein the composition is effective to reduce at least one symptom of the cancer in the subject.

The present invention provides in certain embodiments a kit comprising at least one anti-cancer therapy and a pharmaceutical composition comprising a pharmaceutically acceptable carrier and an amount of an IL-1α inhibitor, a container, and a package insert or label indicating the administration of the anti-cancer therapy and the IL-1α inhibitor, for reducing at least one symptom of the cancer in the subject.

### BRIEF DESCRIPTION OF DRAWINGS

FIGS. 1A-D. Network analysis of erlotinib-treated SQ20B cells. A-C: Shown are the 3 significant networks constructed from differentially regulated transcripts comparing microarray data from Erlotinib (5 μM, 48 h) treated SQ20B head and neck squamous carcinoma (HNSCC) cells versus DMSO treated HNSCC cells. The microarray expression value changes were uploaded to and analyzed by MetaCore™ (GeneGo) software with thresholds set at 1.5 and p value <0.05. Up regulated genes are marked with red circles; down regulated with blue circles. The ‘checkerboard’ color indicates mixed expression for the gene between cell lines. D: GO Processes within this network are listed with percentage of genes affected and relevant p values.

FIGS. 2A-I. Role of TLR signaling in erlotinib-induced IL-6 in HNSCC cells. A,B: RNA isolated from two HNSCC tumors (#9 (A) and #13 (B)) and matched normal tissue was analyzed for TLR expression by RTPCR. C: SQ20B, Cal27 and FaDu cells were treated with the following TLR agonists for 48 hours: Pam3CSK4 (200 ng/mL; TLR1/2); FSL (100 ng/mL; TLR2/6); Poly I:C (20 μg/mL; TLR3); LPS (200 ng/mL; TLR4); Flagellin (200 ng/mL; TLRS); Gardiquimod (1 μg/mL; TLR7); CL075 (1 μg/mL; TLR7/8); E. Coli DNA (1 μg/mL; TLR9). Secreted IL-6 was measured by ELISA. D-I: SQ20B (D,E,H,I) and Cal-27 (F,G) cells were transfected with scrambled siRNA control (siCON), siRNA targeted against TLR2 (siTLR2) (D-G), or siRNA targeted against TLRS (siTLR5) (H,I). Cells were treated with DMSO (black bars) or erlotinib (5 μM; gray bars) for 48 hours and secreted IL-6 measured by ELISA. IL-6 values were normalized to cell number and reported as fold change over siCON. Knockdown of respective TLR was confirmed by RTPCR (G,H). n=3, errors bars=SEM. *p<0.05 versus control; **p<0.05 versus ERL.

FIGS. 3A-J: Role of IL-1R in erlotinib-induced IL-6 secretion. A-B: Normal tissue and matched human HNSCC tumors were analyzed for expression of IL-1 and IL-18 pathway components by RTPCR. Tumor #9 (A) and Tumor #13 (B) are shown with matched normal tissue. C-D: SQ20B (C) and Ca127 (D) cells were treated with IgG control or an IL-18R neutralizing antibody for two hours prior to a 48-hour treatment of DMSO (black bars) or erlotinib (5 μM; gray bars). E-F: SQ20B (E) and Ca127 (F) cells were treated with water (CON) or 50 ng/mL and 10 ng/mL respectively of anakinra (ANA) for two hours followed by 48 hour treatment with DMSO (black bars) or erlotinib (5 μM; gray bars). G-J: SQ20B cells were transfected with a plasmid containing shRNA targeted against IL-1R1 (shIL1R), or a control plasmid (shCON), and selected with zeocin. Clones were analyzed for IL-1R1 levels by RTCPR (G) and Western blot (H). I: Selected clones were analyzed for IL-6 levels and clonogenic survival in the absence and presence of erlotinib (J). IL-6 levels were measured by ELISA and values were normalized to cell number and reported as fold change over CON. n=3, errors bars=SEM. *p<0.05 versus control; **p<0.05 versus ERL.

FIGS. 4A-I: Erlotinib increases IL-1α secretion. (A) SQ20B and (B) Cal27 cell lines were treated with DMSO (black bars) or erlotinib (gray bars) for indicated time points and analyzed for IL-1α. SQ20B (C) and Cal27 (D) cells were treated with water or 1 ng/mL recombinant IL-1α for two hours, then treated with DMSO (black bars) or erlotinib (5 μM; gray bars) for 48 hours, then analyzed for IL-6 secretion. SQ20B (E) and Cal27 (F) cells were treated with anti-IL-1α or anti-IL-1β neutralizing antibodies for two hours prior to treatment with DMSO (black bars) or erlotinib (gray bars) for 48 hours then analyzed for IL-6. G: SQ20B cells were treated with neutralizing antibodies were used to perform a clonogenic assay. H: SQ20B cells were treated with Z-VAD-fmk, a pan-caspase inhibitor or Y-VAD-fmk, a caspase 1 inhibitor for one hour prior to 48-hour DMSO (black bars) or erlotinib (gray bars) treatment then analyzed for IL-1α. I: SQ20B cells were treated with Z-VAD with or without erlotinib and analyzed for cell survival by clonogenic assay. IL-6 and IL-1α were measured by ELISA, normalized to cell number, and reported as fold change over CON. n=3, error bars=SEM *p<0.05 versus control; **p<0.05 versus ERL.

FIGS. 5A-F: IL-1α expression is negatively correlated with survival in HNSCC. A dataset (N=88) of HNSCC tumors (The Cancer Genome Atlas) was analyzed for MyD88-dependent receptor expression such as TLRs (A), IL-18R (B) and IL-1R (C) and expression of IL-1 ligands such as IL-1α (D), IL-1RN (E) and IL-1β (F). The highest quartile of expressing tumors was plotted against the lowest quartile in Kaplan-Meier survival curves. P-values were calculated with Log-rank (Mantel-Cox) test.

FIGS. 6A-F: Stable knockdown of MyD88 reduces IL-6 and tumor growth in a xenograft model of HNSCC. SQ20B cells were transfected with a plasmid containing shRNA targeted against MyD88 (shMyD88), or a control plasmid (shCON), and selected with zeocin. (A) Clones were analyzed for MyD88 levels by Western blot (A) and secreted IL-6 levels by ELISA in the absence (black bars) and presence (gray bars) of 5 μM erlotinib (B). C-F: SQ20B cells with stable knockdown of MyD88 (shMyD88 #2 and shMyD88 #9) or a control plasmid (shCON) were injected into the right flank of athymic nu/nu mice (2x107 cells per mouse). shMyD88 clone #2 (C) and shMyD88 clone #9 (E) were measured for tumor growth compared to control over a three week treatment period (12.5 mg/kg erlotinib or water daily). Tumor volume at Day 17 is shown for clone #2 (D) and clone #9 (F). (n=11-13, error bars=SEM).

FIGS. 7A-D. Process network and disease analyses of erlotinib-treated HNSCC cells. Shown are the top ten upregulated cellular/molecular processes (A,B) and diseases (C,D) from differentially regulated transcripts comparing microarray data from erlotinib (5 μM, 48 h) treated SQ20B (A,C) and Cal-27 (B,D) head and neck squamous carcinoma cells versus DMSO treated cells.

FIGS. 8A-D. Pathway and network analysis of erlotinib-treated HNSCC cells. Shown are the top ten upregulated pathways (A,B) and top upregulated inflammation-related networks (C,D) constructed from differentially regulated transcripts comparing microarray data from erlotinib (5 μM, 48 h) treated SQ20B (A,C) and Cal-27 (B,D) head and neck squamous carcinoma cells versus DMSO treated cells. Up regulated genes are marked with red circles; down regulated with blue circles. The ‘checkerboard’ color indicates mixed expression for the gene between cell lines.

FIGS. 9A-G: Knockdown of MyD88 reduces IL-6 and tumor growth in HNSCC cells. SQ20B (A) and Cal-27 (B) cells were transfected with scrambled siRNA control (siCON), or siRNA targeted against MyD88 (siMyD88). Cells were treated with DMSO (black bars) or erlotinib ([ERL], 5 μM; gray bars) for 48 hours and IL-6 measured by ELISA. SQ20B cells were transfected with a shRNA targeted against MyD88 (shMyD88), or a control plasmid (shCON), and selected with zeocin. Clones were analyzed for MyD88 levels by Western blot (C inset) and IL-6 in the presence of DMSO and 5 μM ERL (C). D-G: The above clones were injected into the right flank of athymic nu/nu mice. Tumor growth was measured over a three week treatment period (12.5 mg/kg ERL or water daily) (D,E). Tumor volume at Day 17 is shown for clone #2 (F) and clone #9 (G). N=11-13. Error bars=standard error of the mean (SEM). *p<0.05 versus control; **p<0.05 versus ERL.

FIGS. 10A-J. Role of TLR signaling in erlotinib-induced IL-6 in HNSCC cells. A,B: RNA isolated from two HNSCC tumors (#9 (A) and #13 (B)) (gray bars) and matched normal tissue (black bars) was analyzed for TLR1-10, IL-1R and IL-18R gene expression by RTPCR. C: SQ20B, Cal27 and FaDu cells were treated with TLR agonists as described in the Methods section. Secreted IL-6 was measured by ELISA. D: SQ20B and Cal-27 were treated with DMSO or 5 μM erlotinib (ERL) for 48 hours. Cells were analyzed by RTPCR for expression of TLR genes. Values were normalized to 18S mRNA levels, and reported as fold change over DMSO (set at 1, dotted line). E-H: SQ20B or Cal-27 cells were transfected with scrambled siRNA control (siCON), siRNA targeted against TLR2 (siTLR2) (E,F), or siRNA targeted against TLR5 (siTLR5) (G,H), treated with DMSO or 5μM ERL, then analyzed for IL-6. Knockdown of respective TLRs were confirmed by RTPCR (F,H). SQ20B (I) and Cal27 (J) cells were treated with IgG or an IL-18R neutralizing antibody (nIL-18Rab, 0.5 ug/mL) for two hours prior to DMSO or ERL (5 μM) before IL-6 analysis. N=3, errors bars=standard error of the mean (SEM). *p<0.05 versus control; **p<0.05 versus ERL.

FIGS. 11A-G: Role of IL-1 signaling in erlotinib-induced IL-6 secretion. SQ20B (A) and Ca127 (B) cells were treated with DMSO (CON) or 50 ng/mL and 10 ng/mL respectively of anakinra (IL-1RA) for two hours followed by 48 hour treatment with DMSO or erlotinib ([ERL], 5 μM) then analysis for IL-6 secretion by ELISA. C: SQ20B cells were transfected with shRNA targeted against IL-1R1 (shIL-1R1), or a control plasmid (shCON/shGFP), and selected with zeocin. Clones were analyzed for IL-1R1 levels by western blot (C inset) and IL-6 levels. A dataset (n=41) of HNSCC tumors (T) and matched normal tissue (N) from The Cancer Genome Atlas was analyzed for expression of IL-1α, IL-1β, and IL-1RA mRNA. Linear fold change (tumor over normal) is reported (D). E: Cell lines were treated with DMSO or 5μM ERL for the indicated time points and analyzed for IL-1α by ELISA. F: Cells were treated with PBS or 1 ng/mL human recombinant IL-1α for two hours, treated with DMSO or ERL, then analyzed for IL-6 secretion. G: Cells were treated with anti-IL-1α or anti-IL-1β neutralizing antibodies for two hours prior to treatment with DMSO or ERL then analyzed for IL-6. N=3, errors bars=SEM. *p<0.05 versus control; **p<0.05 versus ERL.

FIGS. 12A-F: Erlotinib increases IL-1α secretion via oxidative stress-mediated cell death. A: SQ20B and Cal-27 cells were pre-treated with Z-VAD-fmk (ZVAD) or Y-VAD-fmk (YVAD) for one hour prior to 48-hour DMSO or 5 μM erlotinib (ERL) then analyzed for IL-1α by ELISA. B,C: SQ20B (B) and Cal-27 (C) cells were pre-treated with 20 mM N-acetyl cysteine (NAC) or 100 U/mL pegylated catalase (CAT) for 1 h before treatment with ERL, then analyzed for IL-1α and IL-6 secretion by ELISA. D-F: SQ20B (D) and Cal-27 (E) were transfected with empty (EMP), wildtype NADPH oxidase-4 (N4wt) or dominant negative NOX4 (N4dn) adenoviral vectors before treatment with DMSO or ERL, then analysis for IL-1α and IL-6 secretion by ELISA (D,E) and clonogenic survival (F). N=3, errors bars=SEM. *p<0.05 versus control; **p<0.05 versus ERL.

FIGS. 13A-J: IL-1α expression affects response to EGFR inhibitors in HNSCC. A dataset (N=88) of HNSCC tumors from The Cancer Genome Atlas was analyzed for MyD88 (A), TLRs (B), IL-18R (C), IL-1R (D), IL-1α (E), IL-1β (G) and IL-1RN (H) expression. A dataset (N=48) of HNSCC tumors from patients that received targeted molecular therapy (TMT) was also analyzed for IL-1α expression (F). The highest quartile of expressing tumors was plotted against the lowest quartile in Kaplan-Meier survival curves. SQ20B cells were treated with IL-1α (anti-IL-1a) or IL-1β (anti-IL-1β) neutralizing antibodies for two hours prior to treatment with DMSO (black bars) or erlotinib (ERL, 5 uM, gray bars) for 48 hours then analyzed for clonogenic survival, n=3 (I). J: Schematic representing the proposed role of IL-1 signaling in the reduced effect of ERL in HNSCC. Error bars represent the standard error of the mean (SEM). *p<0.05 versus control; **p<0.05 versus ERL.

### DETAILED DESCRIPTION OF THE INVENTION

Anti-Cancer Therapies

In certain embodiments, the at least one anti-cancer therapy is radiation therapy.

In certain embodiments, the at least one anti-cancer therapy is immunotherapy.

In certain embodiments, the at least one anti-cancer therapy is a chemotherapeutic agent. In certain embodiments, the chemotherapeutic agent is an epidermal growth factor receptor (EGFR) inhibitor. In certain embodiments, the EGFR inhibitor is Erlotinib, lapatinib, cetuximab or panitumumab. In certain embodiments, the EGFR inhibitor is Erlotinib. In certain embodiments, the chemotherapeutic agent is Methotrexate (Abitrexate, Folex, Methotrexate LPF, Mexate, or Mexate-AQ), Fluorouracil (Adrucil, Fluoroplex, or Efudex), Bleomycin (Blenoxane), Cisplatin (Platinol, Platinol-AQ), Docetaxel (Taxotere), carboplatin (Paraplatin, Paraplatin-AQ) and/or paclitaxel (Abraxane, Taxol).

In certain embodiments, the cancer therapeutic agent is a drug combination used in head and neck cancer, such as docetaxel, cisplatin, and fluorouracil (TPF).

Interleukin-1 Alpha Inhibitors

In certain embodiments, the IL-1α inhibitor is an anti-IL-1α monoclonal antibody (mAb) or a fragment of an anti-IL-1α mAb (e.g., a complementarity determining region of an anti-IL-1αmAb). In certain embodiments, the IL-1α inhibitor is anakinra.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. Thus, as used herein, an “antibody” or “Ab” is an immunoglobulin (Ig), a solution of identical or heterogeneous Igs, or a mixture of Igs. An “Ab” can also refer to fragments and engineered versions of Igs such as Fab, Fab′, and F(ab′)₂ fragments; and scFv's, heteroconjugate Abs, and similar artificial molecules that employ Ig-derived CDRs to impart antigen specificity. A “mAb” or “mAb” is an Ab expressed by one clonal B cell line or a population of Ab molecules that contains only one species of an antigen binding site capable of immunoreacting with a particular epitope of a particular antigen. A “polyclonal Ab” or “polyclonal Ab” is a mixture of heterogeneous Abs. Typically, a polyclonal Ab will include myriad different Ab molecules which bind a particular antigen with at least some of the different Abs immunoreacting with a different epitope of the antigen. As used herein, a polyclonal Ab can be a mixture of two or more mAbs.

An “antigen-binding portion” of an Ab is contained within the variable region of the Fab portion of an Ab and is the portion of the Ab that confers antigen specificity to the Ab (i.e., typically the three-dimensional pocket formed by the CDRs of the heavy and light chains of the Ab). A “Fab portion” or “Fab region” is the proteolytic fragment of a papain-digested Ig that contains the antigen-binding portion of that Ig. A “non-Fab portion” is that portion of an Ab not within the Fab portion, e.g., an “Fc portion” or “Fc region.” A “constant region” of an Ab is that portion of the Ab outside of the variable region. Generally encompassed within the constant region is the “effector portion” of an Ab, which is the portion of an Ab that is responsible for binding other immune system components that facilitate the immune response. Thus, for example, the site on an Ab that binds complement components or Fc receptors (not via its antigen-binding portion) is an effector portion of that Ab.

An “isolated” or “purified” antibody is one which has been separated from a component of its natural environment. Typically, an Ab or protein is purified when it is at least about 10% (e.g., 9%, 10%, 20%, 30% 40%, 50%, 60%, 70%, 80%, 90%, 95%, 98%, 99%, 99.9%, and 100%), by weight, free from the non-Ab proteins or other naturally-occurring organic molecules with which it is naturally associated. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007). A chemically-synthesized protein or other recombinant protein produced in a cell type other than the cell type in which it naturally occurs is “purified

[symbol]

The terms anti-polypeptide of interest antibody and “an antibody that binds to” a polypeptide of interest refer to an antibody that is capable of binding a polypeptide of interest with sufficient affinity such that the antibody is useful as a diagnostic and/or therapeutic agent in targeting a polypeptide of interest. In one embodiment, the extent of binding of an anti-polypeptide of interest antibody to an unrelated, non-polypeptide of interest protein is less than about 10% of the binding of the antibody to a polypeptide of interest as measured, e.g., by a radioimmunoassay (RIA). In certain embodiments, an antibody that binds to a polypeptide of interest has a dissociation constant (Kd) of ≦1 μM, ≦100 nM, ≦10 nM, ≦1 nM, ≦0.1 nM, ≦0.01 nM, or ≦0.001 nM (e.g., 10⁻⁸ M or less, e.g., from 10⁻⁸ M to 10⁻¹³ M, e.g., from 10⁻⁹ M to 10¹³ M). In certain embodiments, an anti-polypeptide of interest antibody binds to an epitope of a polypeptide of interest that is conserved among polypeptides of interest from different species.

A “blocking antibody” or an “antagonist antibody” is one which inhibits or reduces biological activity of the antigen it binds. Preferred blocking antibodies or antagonist antibodies substantially or completely inhibit the biological activity of the antigen.

“Affinity” refers to the strength of the sum total of noncovalent interactions between a single binding site of a molecule (e.g., an antibody) and its binding partner (e.g., an antigen).

Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv); and multispecific antibodies formed from antibody fragments.

The term “chimeric” antibody refers to an antibody in which a portion of the heavy and/or light chain is derived from a particular source or species, while the remainder of the heavy and/or light chain is derived from a different source or species.

The terms “full length antibody,” “intact antibody,” and “whole antibody” are used herein interchangeably to refer to an antibody having a structure substantially similar to a native antibody structure or having heavy chains that contain an Fc region.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

Conditions Treated

In certain embodiments, the cancer is a hematopoietic cancer.

In certain embodiments, the cancer is a solid tumor. In certain embodiments, the cancer is a HNSCC tumor. In certain embodiments, the tumor is decreased in size in the subject by at least about 10% (e.g., at least 8, 9, 10, 15, 17, 20, 30, 40, 50, 60, 70, 80, 90, or 100%).

Pharmaceutical Compositions

In certain embodiments, the present invention provides a pharmaceutical composition for treating cancer in a subject comprising a pharmaceutically acceptable carrier, an amount of chemotherapeutic agent, and an amount of an IL-1α inhibitor, wherein the composition is effective to reduce at least one symptom of the cancer in the subject.

In certain embodiments, the chemotherapeutic agent is an epidermal growth factor receptor (EGFR) inhibitor. In certain embodiments, the EGFR inhibitor is Erlotinib, lapatinib, cetuximab or panitumumab. In certain embodiments, the EGFR inhibitor is Erlotinib.

In certain embodiments, the chemotherapeutic agent is Methotrexate (Abitrexate, Folex, Methotrexate LPF, Mexate, or Mexate-AQ), Fluorouracil (Adrucil, Fluoroplex, or Efudex), Bleomycin (Blenoxane), Cisplatin (Platinol, Platinol-AQ), and/or Docetaxel (Taxotere).

In certain embodiments, the IL-1α inhibitor is an anti-IL-1α monoclonal antibody (mAb). In certain embodiments, the IL-1α inhibitor is anakinra.

Administration of Therapeutic Agent

The term “therapeutically effective amount,” in reference to treating a disease state/condition, refers to an amount of a compound either alone or as contained in a pharmaceutical composition that is capable of having any detectable, positive effect on any symptom, aspect, or characteristics of a disease state/condition when administered as a single dose or in multiple doses. A “therapeutically effective amount” is an amount which is capable of producing a medically desirable effect in a treated animal or human (e.g., amelioration or prevention of a disease or symptom of a disease). Such effect need not be absolute to be beneficial.

A therapeutically effective amount is an amount which is capable of producing a medically desirable result in a treated animal or human. An effective amount of anti-IL-1a Ab compositions and anti-cancer therapy is an amount which shows clinical efficacy in patients as measured by the improvement in one or more a tumor-associated disease characteristics described above. As is well known in the medical arts, dosage for any one animal or human depends on many factors, including the subject's size, body surface area, age, the particular composition to be administered, sex, time and route of administration, general health, and other drugs being administered concurrently. Preferred doses range from about 0.2 to 20 (e.g., 0.15, 0.2, 0.3, 0.4, 0.5, 1, 2, 3, 4, 5, 6, 7, 8, 9, 10, 15, 20, 30, 50, or 100) mg/kg body weight. The dose may be given repeatedly, e.g., hourly, daily, semi-weekly, weekly, bi-weekly, tri-weekly, or monthly.

The terms “treat,” “treating” and “treatment” as used herein include administering a compound prior to the onset of clinical symptoms of a disease state/condition so as to prevent any symptom, as well as administering a compound after the onset of clinical symptoms of a disease state/condition so as to reduce or eliminate any symptom, aspect or characteristic of the disease state/condition. “Treating” as used herein refers to ameliorating at least one symptom of, curing and/or preventing the development of a given disease or condition. Such treating need not be absolute to be useful.

The pharmaceutical composition can be administered to the subject by injection, subcutaneously, intravenously, intramuscularly, or directly into a tumor. In the method, the dose can be at least 0.25 (e.g., at least 0.2, 0.5, 0.75, 1, 2, 3, 4, or 5) mg/ml.

In certain embodiments, the IL-1α inhibitor is administered prior to the administration of the anti-cancer therapy.

In certain embodiments, the IL-1α inhibitor and/or the anti-cancer therapy is administered parenterally. In certain embodiments, the IL-1α inhibitor and/or the anti-cancer therapy is administered intramuscularly, subcutaneously, intradermally or intravenously. In certain embodiments, the IL-1α inhibitor and/or the anti-cancer therapy is administered orally or intranasally.

In certain embodiments, the IL-1α inhibitor and the anti-cancer therapy are administered simultaneously.

In certain embodiments, the IL-1α inhibitor and the anti-cancer therapy are administered sequentially.

In certain embodiments, the administration of the IL-1α inhibitor begins about 1 to about 10 days before administration of the anti-cancer therapy.

In certain embodiments, the administration of the anti-cancer therapy begins about 1 to about 10 days before administration of the IL-1α inhibitor.

In certain embodiments, the administration of the IL-1α inhibitor and the anti-cancer therapy begin on the same day.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions that can be used to deliver the compounds of the present invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compounds of the present invention can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

The compound is conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Kits

In certain embodiments, the present invention provides a kit comprising at least one anti-cancer therapy and a pharmaceutical composition comprising a pharmaceutically acceptable carrier and an amount of an IL-1α inhibitor, a container, and a package insert or label indicating the administration of the anti-cancer therapy and the IL-1α inhibitor, for reducing at least one symptom of the cancer in the subject.

Although methods and materials similar or equivalent to those described herein can be used in the practice or testing of the present invention, suitable methods and materials are described below. All publications mentioned herein are incorporated by reference in their entirety. In the case of conflict, the present specification, including definitions will control. In addition, the particular embodiments discussed below are illustrative only and not intended to be limiting.

### EXAMPLE 1

Abstract

Epidermal growth factor receptor (EGFR) is upregulated in the majority of head and neck squamous cell carcinomas (HNSCC). However many HNSCC patients respond poorly to EGFR inhibitors (EGFRIs) despite tumor expression of EGFR. Gene expression analysis of erlotinib-treated HNSCC cell lines revealed an upregulation of genes involved in MyD88-dependent interleukin-6 (IL-6) expression compared to their respective vehicle treated cell lines. We therefore proposed that MyD88-dependent signaling may reduce the anti-tumor efficacy of the EGFR inhibitor erlotinib in HNSCC. Erlotinib significantly upregulated IL-6 secretion but there was little to no evidence of toll-like receptor (TLR) or interleukin-18 receptor (IL-18R) involvement. However, suppression of interleukin-1 receptor (IL-1R) signaling through pharmacologic and genetic methods significantly reduced erlotinib-induced IL-6 production and increased HNSCC cell sensitivity to erlotinib in vitro. A time-dependent increase of IL-1 alpha (IL-1α) but not IL-1 beta (IL-1β3) was observed in response to erlotinib treatment and pre-treatment with a pan-caspase inhibitor but not a caspase-1 inhibitor reduced erlotinib-induced IL-1α secretion. Human HNSCC tumors showed higher IL-1α mRNA levels compared to matched normal tissue, and IL-1α was found to be negatively correlated with survival in HNSCC patients. Lastly, suppression of MyD88 expression significantly blocked erlotinib-induced IL-6 secretion in vitro and increased the anti-tumor activity of erlotinib in vivo. Overall, the IL-1α/IL-1R/MYD88/IL-6 pathway may be responsible for the reduced anti-tumor efficacy of erlotinib and other EGFRIs; and blockade of the MyD88-dependent signaling may improve the efficacy of EGFRIs in the treatment of HNSCC.

Introduction

The epidermal growth factor receptor (EGFR) is a receptor tyrosine kinase that activates numerous pro-survival signaling pathways (1). Given that EGFR signaling is upregulated in many cancers especially head and neck squamous cell carcinoma (HNSCC), several drugs that target EGFR have been developed and approved for cancer therapy (1). However, response rates to EGFR inhibitors (EGFRIs) are quite poor and this is evident especially in HNSCC patients with recurrent or metastatic (RIM) disease (2-5). Many different mechanisms (e.g. existing/acquired mutations and alternative signaling pathways) have been proposed that may induce resistance or affect patient response to EGFRIs, but this knowledge has not improved survival rates for HNSCC patients to date (6-9).

Previous studies in our laboratory observed a significant upregulation in IL-6 expression in three HNSCC tumor cell lines treated with EGFRIs such as erlotinib, lapatinib, cetuximab and panitumumab (10). IL-6 is a pleotropic cytokine with a wide range of biological activities and is well known for its role in inflammation, tumor progression and chemoresistance in HNSCC (11-14). We additionally demonstrated the ability of IL-6 signaling to protect HNSCC against erlotinib treatment in vitro and in vivo (10) suggesting that IL-6 may be involved in resistance to EGFRIs.

A well-established mechanism of IL-6 production involves the cytosolic adaptor protein MyD88, which acts through intermediaries to induce NFKB activation (15). MyD88 is required for the activity of members of the Toll/Interleukin-1 receptor (TIR) superfamily which include Toll-like Receptors (TLRs), the Interleukin-1 Receptor (IL-1R), and the IL-18 Receptor (IL-18R) (15). Activation of TIR superfamily members lead to the recruitment of MyD88 via its TIR domain which in turn recruits members of the IRAK family leading to downstream NFkB activation and expression of pro-inflammatory cytokines including IL-6 (15). Here we show that EGFR inhibition using the EGFR tyrosine kinase inhibitor erlotinib activates the IL-1R/MyD88/IL-6 signaling pathway and this pathway may serve as a novel mechanism responsible for the poor long-term anti-tumor efficacy of EGFR inhibitors in HNSCC therapy.

Materials and Methods

Cells and Culture Conditions:

Cal-27 and FaDu human head and neck squamous carcinoma (HNSCC) cells were obtained from the American Type Culture Collection (ATCC, Manassas, Va.). SQ20B HNSCC cells (16) were a gift from Dr. Anjali Gupta (Department of Radiation Oncology, The University of Iowa). All HNSCC cell lines are EGFR positive and are sensitive to EGFR inhibitors. All cell lines were authenticated by the ATCC for viability (before freezing and after thawing), growth, morphology and isoenzymology. Cells were stored according to the supplier's instructions and used over a course of no more than 3 months after resuscitation of frozen aliquots. Cultures were maintained in 5% CO₂ and air humidified in a 37° C. incubator.

Drug Treatment:

Erlotinib (ERL; Tarceva) and anakinra (ANA; Kineret) were obtained from the inpatient pharmacy at the University of Iowa Hospitals and Clinics. Drugs were added to cells at final concentrations of 5 μM ERL and 10 ng/mL or 50 ng/mL ANA. Human IgG and dimethyl sulfoximine (DMSO) were used as controls and were obtained from Sigma Aldrich. Human IL-1α, IL-1β, and IL-18Rα neutralizing antibodies were obtained from R&D Systems and were used at a concentration of 0.5 μg/mL. Recombinant human IL-1α was obtained from Life Technologies and administered at a concentration of 1 ng/mL. Ac-Y-VAD-cho, a caspase-1 inhibitor (CalBioChem), was suspended in DMSO and added at a concentration of 5 Z-VAD-fmk, a pan-caspase inhibitor (Promega) was diluted in DMSO and added at a concentration of 20 μM. TLR agonists were used at the following concentrations: Pam3CSK4 (200ng/mL), FSL-1 (100 ng/mL), Poly I:C (20 μg/mL), LPS (200 ng/mL), Flagellin (200 ng/mL), Gardiquimod (1 μg/mL), CL075 (1 μg/mL), and E. coli DNA (1 μg/mL). All TLR agonists were obtained from InvivoGen. The required volume of each drug was added directly to complete cell culture media on cells to achieve the indicated final concentrations.

Enzyme-Linked Immunosorbent Assay:

Levels of IL-6, IL-1α and IL-1β of treated cells were determined by ELISA. The culture media of the treated cells were harvested and each cytokine was detected according to the manufacturer's protocol using the Human Quantikine ELISA Kits (R&D Systems, Minneapolis, Minn.).

Western Blot Analysis:

Cell lysates were standardized for protein content, resolved on 4% to 12% SDS polyacrylamide gels, and blotted onto nitrocellulose membranes. Membranes were probed with rabbit anti-MyD88 (1:500, Cell Signaling), anti-IL-1R1 (1:500, Santa Cruz), anti-beta-actin (1:5000, Thermo Scientific). Antibody binding was detected by using an ECL Chemiluminescence Kit (Amersham).

siRNA Transfection:

TLR2, TLRS and control siRNA (Santa Cruz) were transfected into HNSCC cells at a concentration of 40-80 nM with equal volume Lipofectamine RNAiMAX (Invitrogen). Cells were incubated in Opti-MEM for 4 hours prior to addition of siRNA and 16 hours after addition of siRNA. Cells were allowed to recover 48-72 hours in antibiotic-free DMEM with 10% FBS before 48-hour erlotinib treatment. Knockdown was confirmed by RT-PCR and/or western blot.

shRNA Transfection:

Low-passage SQ20B cells were transfected with 1 μg/mL of ready-made psiRNA-h7SKGFPzeo (control plasmid), psiRNA-shMyD88, or psiRNA-shIL1R (Invivogen) in the presence of Opti-MEM and Lipofectamine RNAiMAX as detailed above. After transfection, cells were allowed to recover for 48 hours in antibiotic-free DMEM. Zeocin was then added to the media to select for the plasmid, and resulting clones were picked and checked for knockdown by RTPCR and western blot.

Clonogenic Survival Assay:

Clonogenic survival was determined as previously described (17). Individual assays were performed with multiple dilutions with at least four cloning dishes per data point, repeated in at least 3 separate experiments. Tumor cell implantation: Female 4-5 week old athymic-nu/nu nude mice were purchased from Harlan Laboratories (Indianapolis, IN). Mice were housed in a pathogen-free barrier room in the Animal Care Facility at the University of Iowa and handled using aseptic procedures. All procedures were approved by the IACUC committee of the University of Iowa and conformed to the guidelines established by the NIH. Mice were allowed at least 3 days to acclimate prior to beginning experimentation, and food and water were made freely available. Tumor cells (shCON or shMyD88) were inoculated into nude mice by subcutaneous injection of 0.1 mL aliquots of saline containing 2×10⁶ FaDu cells into the right flank using 26-gauge needles.

In Vivo Drugs Administration:

Mice started drug treatment 1 week after tumor inoculation. Mice bearing control (shCON) or MyD88 knockdown (shMyD88) xenograft tumors were subdivided into 2 treatment groups (n=11-14), receiving either 100 μL water or 12.5 mg/kg erlotinib suspended in water. Treatments were given orally every day for three weeks. Mice were evaluated daily and tumor measurements taken three times per week using Vernier calipers. Tumor volumes were calculated using the formula: tumor volume=(length×width²)/2 where the length was the longest dimension, and width was the dimension perpendicular to length. Mice were euthanized via CO₂ gas asphyxiation or lethal overdose of sodium pentobarbital (100 mg/kg) when tumor diameter exceeded 1.5 cm in any dimension.

Statistical Analysis:

Statistical analysis was done using GraphPad Prism version 5 for Windows (GraphPad Software, San Diego, Calif.). Differences between 3 or more means were determined by one-way ANOVA with Tukey post-tests. Linear mixed effects regression models were used to estimate and compare the group-specific change in tumor growth curves. Differences in survival curves were determined by Mantel-Cox test. All statistical analysis was performed at the p<0.05 level of significance.

Results

Network Analysis of Erlotinib-Treated HNSCC Cell Lines

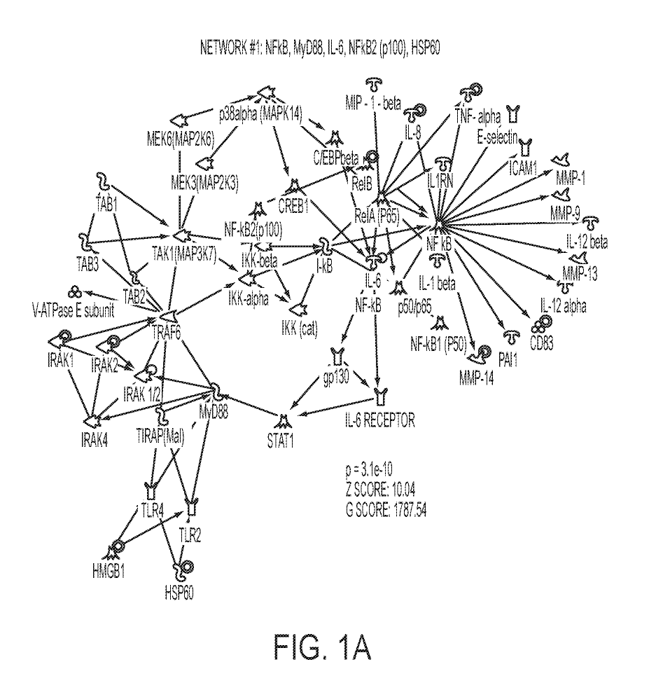

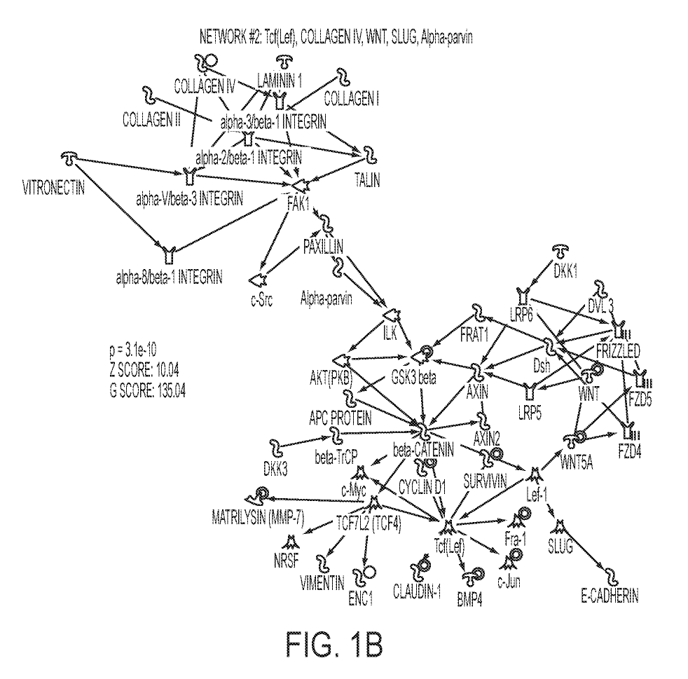

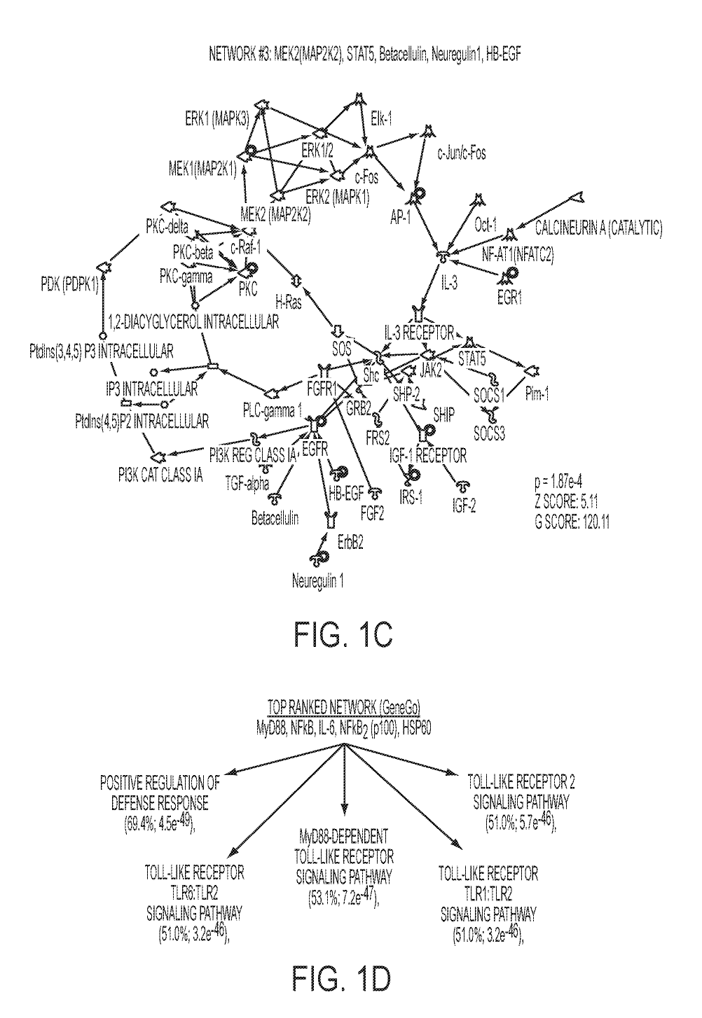

The gene expression profiles of SQ20B HNSCC cells exposed to erlotinib versus DMSO were analyzed by high-throughput microarray. Genetic network analysis of the resultant gene expression data were carried out using Metacore™ (GeneGo) using a threshold of 1.5 in both directions and a p-value of 0.05. Three networks were identified using the GeneGo tool (FIG. 1A-C) that identified functional relationships between gene products based on known interactions in the scientific literature. These networks were identified (in ranking order) as #1: NFkB, MyD88, IL-6, NFkB2 (p100), HSP60 (FIG. 1A); #2: Tcf(Lef), Collagen IV, WNT, SLUG, Alpha-parvin (FIG. 1B); and #3: MEK2(MAP2K2), STATS, Betacellulin, Neuregulin 1, HB-EGF (FIG. 1C). Of these networks, we focused on the top scored network (FIG. 1A). The genes and processes in this network were related to positive regulation of defense response, MyD88-dependent toll-like receptor signaling pathway, toll-like receptor TLR6:TLR2 signaling pathway, toll-like receptor TLR1:TLR2 signaling pathway, and toll-like receptor 2 signaling pathway (FIG. 1D). These analyses pointed to the TLR2/MYD88/IL-6 signaling axis being involved in the mechanism of action of erlotinib.

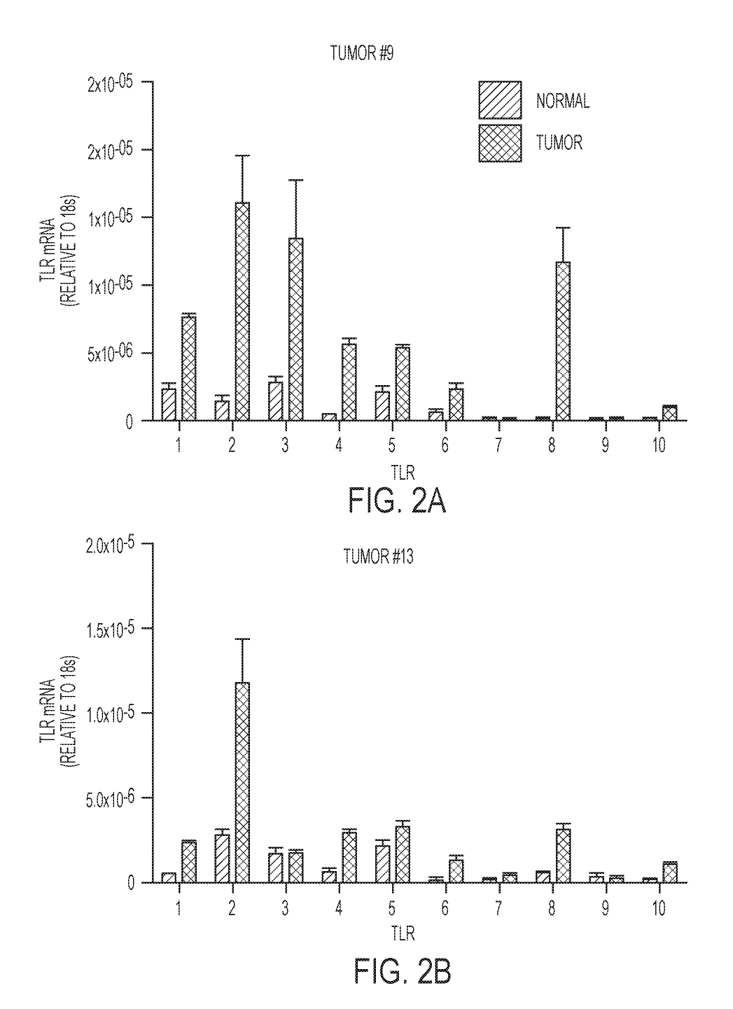

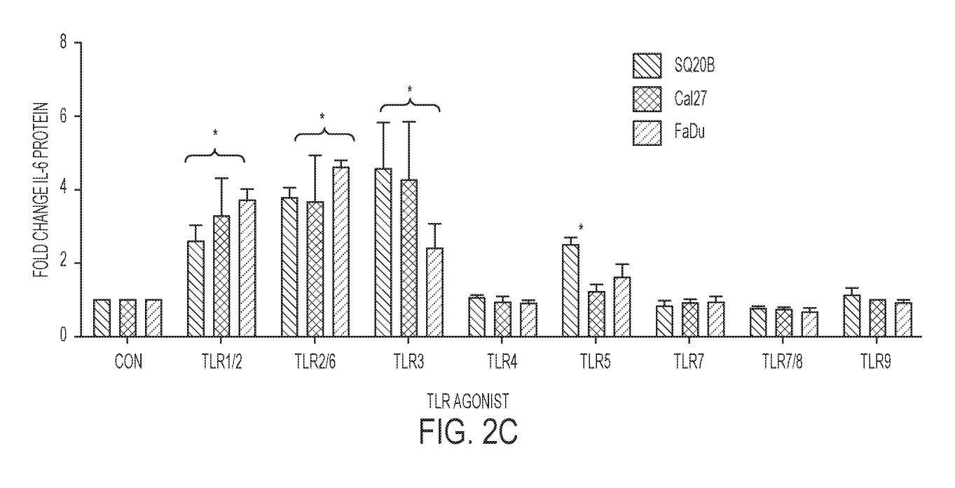

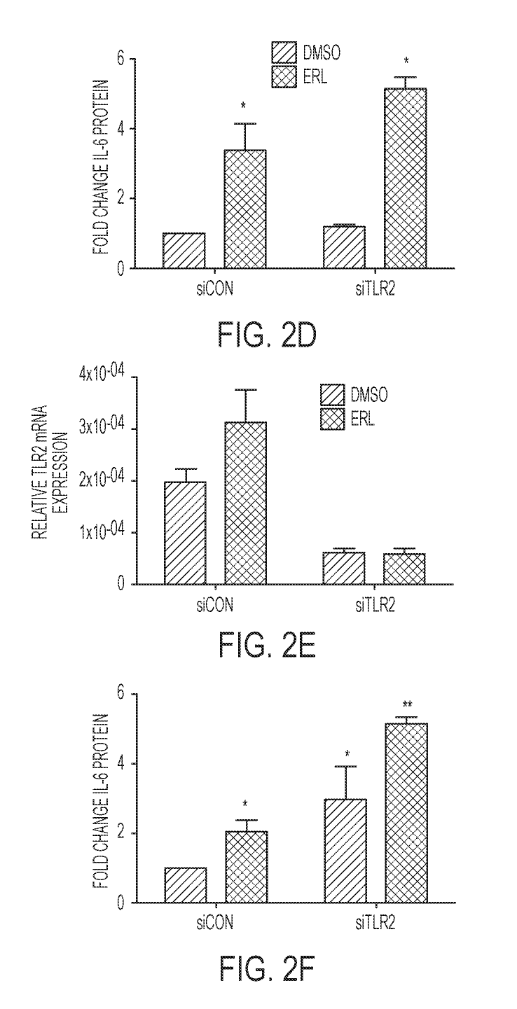

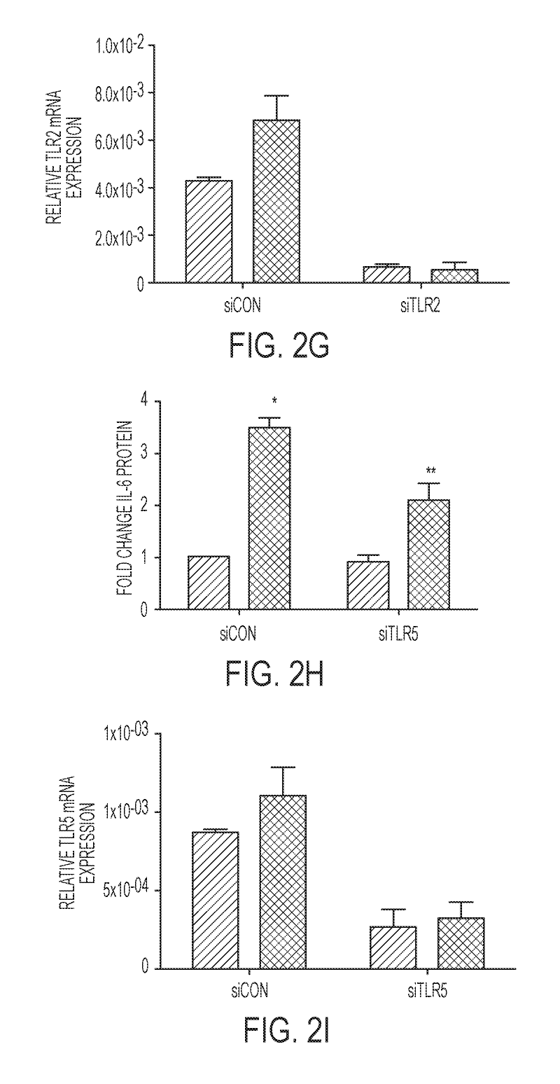

TLR Signaling is not Critical for Erlotinib-Induced IL-6 Secretions Given that MyD88-dependent TLR signaling (specifically TLR2 signaling) was implicated as a top candidate for investigation, we confirmed that human HNSCC tumors and cell lines expressed TLRs. Human HNSCC tumors were obtained from the Tissue Procurement Core (TPC) in the Department of Pathology at the University of Iowa. RT-PCR was used to analyze RNA isolated from these tumors for TLR expression, and found a general trend of increased TLR expression in tumor samples compared to matched normal tissue (FIG. 2A,B). Notably, both tumors showed large increases in expression of TLR2 compared to normal matched tissue (FIG. 2A,B) suggesting that human HNSCC tumors do express TLR2 in addition to other TLRs. In order to confirm that TLRs were expressed and active in our HNSCC cell lines, FaDu, Cal-27 and SQ20B cells were treated with specific TLR agonists, and then analyzed for response to those agonists by assessing IL-6 production. IL-6 secretion was increased after treatment with agonists to TLR1/2, TLR2/6 and TLR3 in all 3 cell lines (FIG. 2C), although TLRS appeared to be active in only the SQ20B cell line (FIG. 2C). As the TLR1/2 and TLR2/6 dimers both depend on TLR2, the activity of these dimers were suppressed using siRNA targeted to TLR2. Erlotinib increases IL-6 production as expected but knockdown of TLR2 expression did not decrease IL-6 in erlotinib treated cells in SQ20B (FIG. 2D,E) and Cal-27 (FIG. 2F,G). However, knockdown of TLRS expression partially but significantly suppressed erlotinib-induced IL-6 secretion in SQ20B cells (FIG. 2H,I). This result was not observed in Cal-27 cells (data not shown). Although TLR3 is not a MyD88-dependent receptor, but rather relies on the adaptor protein TRIF, we additionally confirmed that TLR3 activity was not involved in erlotinib-induced IL-6 in both cell lines by using siRNA targeted to TRIF. Lastly, erlotinib did not affect TLR mRNA expression in Cal-27 or SQ20B cells. Altogether, these results suggest that TLR signaling may not be important for the IL-6 secretion induced by erlotinib although the possible role of TLR5 is currently being further studied.

IL-1R but not IL-18R Signaling is Critical for Erlotinib-Induced IL-6 Expression in HNSCC Cells

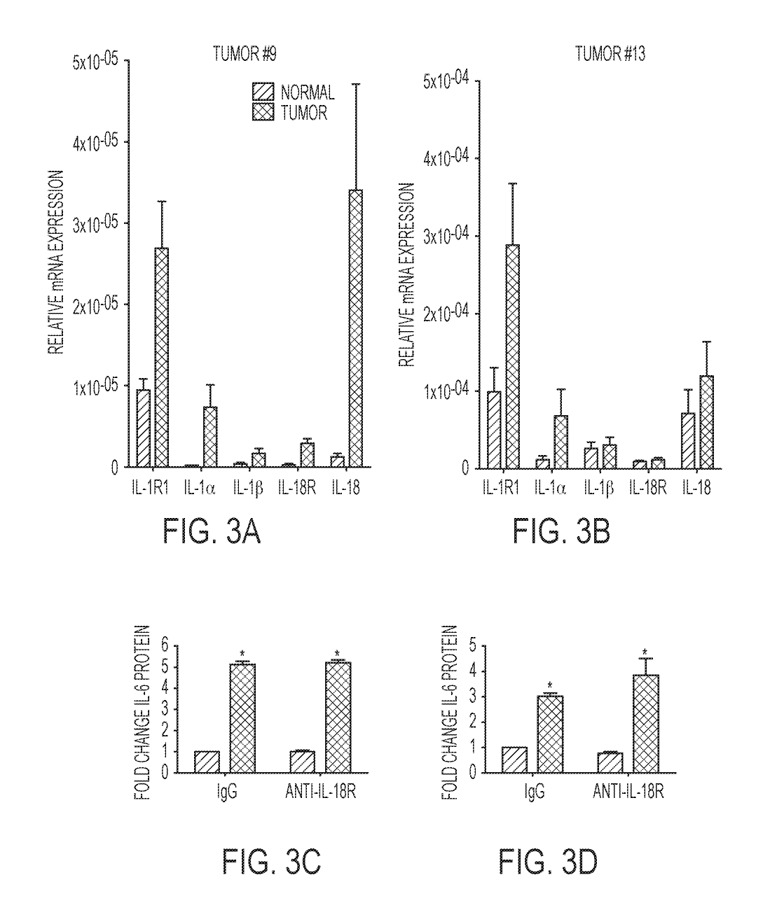

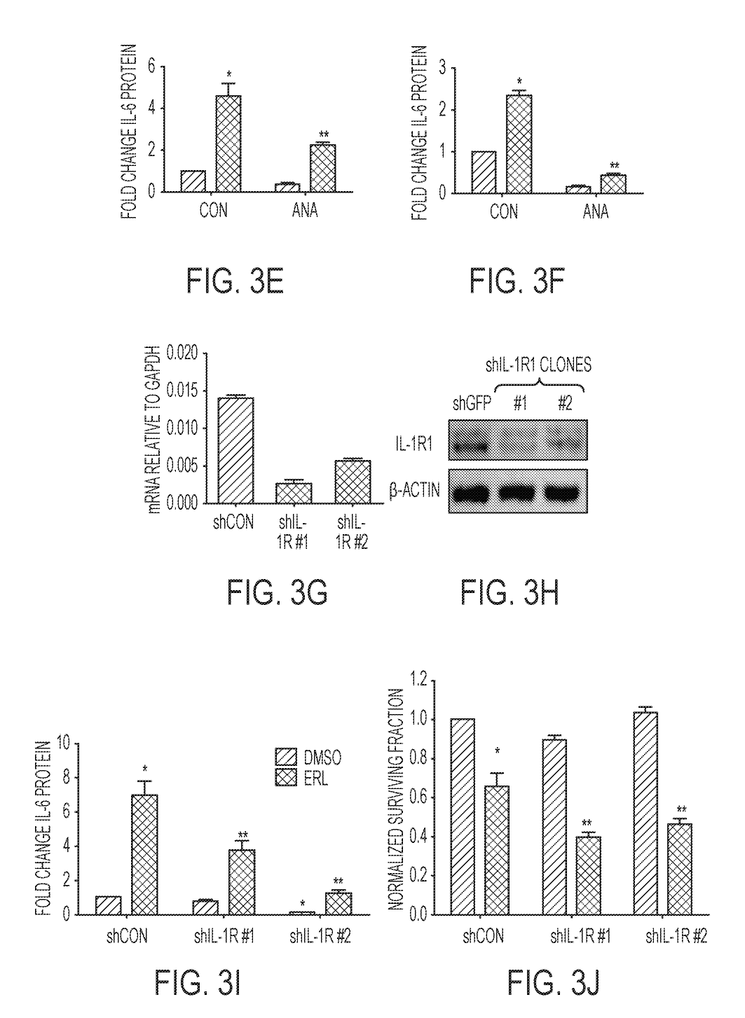

In order to investigate the contribution of other MyD88-dependent signaling pathways, the IL-18R and IL-1R pathways were studied. Human tumors and matched normal tissues which were analyzed in FIG. 2A,B were analyzed again by RT-PCR for specific components of the IL-18 and IL-1 pathways. RNA expression of IL-1R, IL-1α, IL-1β, IL-18R and IL-18 were elevated in tumor #9 compared to normal tissue (FIG. 3A), although only IL-1R and IL-1α were elevated in tumor #13 (FIG. 3B). Pre-treatment of SQ20B and Cal-27 cells for 2 hours with an IL-18R neutralizing antibody prior to erlotinib treatment failed to suppress baseline or erlotinib-induced levels of IL-6 (FIG. 3C,D). However, pretreatment with anakinra, a recombinant IL-1R antagonist (IL-1Ra/IL-1RN) that is FDA approved for use in rheumatoid arthritis, significantly reduced baseline and erlotinib-induced IL-6 in both cell lines (FIG. 3E,F). Additionally, transient knockdown of IL-1 suppressed erlotinib-induced IL-6 in SQ20B and Cal-27 cells and stable knockdown of IL-1R1 (FIG. 3G,H) also led to a decrease in erlotinib-induced IL-6 secretion (FIG. 3I) and increased sensitivity to erlotinib in SQ20B cells in vitro (FIG. 3J). Altogether these results suggest that IL-1R signaling may be involved in erlotinib-induced IL-6.

Erlotinib-Induced Cell Death Triggers IL-1α Release.

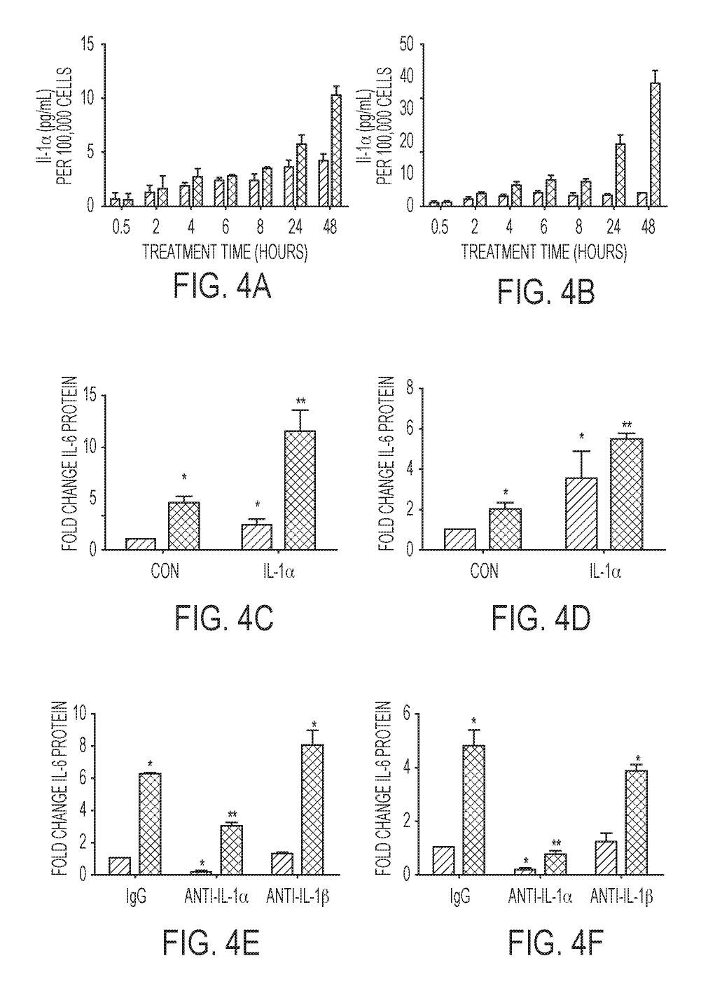

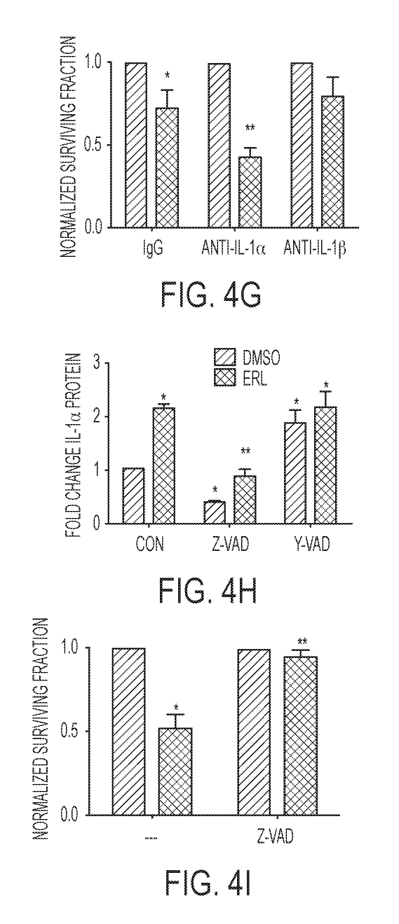

In order to further elucidate the role of IL-1 signaling in erlotinib-induced IL-6 secretion, analysis of the IL-1R ligands IL-1α and IL-1β were carried out using ELISA after erlotinib treatment. IL-1β was undetectable at any time point after erlotinib treatment (data not shown). IL-1α, however, steadily increased across all time points measured in both SQ20B and Ca127 cell lines (FIG. 4A,B). Cal27 cells secreted approximately 5 times more IL-1α than SQ20B, after controlling for cell number (FIG. 4A,B). Administration of exogenous IL-1 increased IL-6 secretion in the presence and absence of erlotinib (FIG. 4C,D) and blockade of IL-1α activity using a IL-1α neutralizing antibody significantly reduced IL-6 secretion in erlotinib-treated cells (FIG. 4E,F). Baseline IL-6 was also significantly decreased in both SQ20B and Cal-27 cells (FIG. 4E,F). Blockade of IL-1β using an IL-β neutralizing antibody had no effect on IL-6 levels in both cell lines (FIG. 4E,F) suggesting that IL-1α release may be responsible for erlotinib-induced IL-6 production. It was further shown that SQ20B cells treated with a IL-1α neutralizing antibody in combination with erlotinib showed a significant reduction in survival compared to the other treatment groups (FIG. 4G) further suggesting that blockade of the IL-1 pathway may increase the sensitivity of erlotinib.

To determine whether cell death is responsible for IL-1α release, we used Z-VAD-fmk, a pan-caspase inhibitor, to prevent cell death. We also tested Ac-Y-VAD-cho, caspase-1 inhibitor, to assess inflammasome involvement. After testing various combinations of these inhibitors, we found an optimal concentration of 20 μM Z-VAD and 5μM Y-VAD. These were the highest doses that did not result in toxicity (increased DMSO percentage resulted in toxicity). In both SQ20B and Cal27 cells, Z-VAD was able to significantly reduce the amount of IL-1α released after erlotinib treatment, as well as baseline levels (FIG. 4H). With caspase-1 inhibition we did not see a decrease in IL-1α levels, and rather saw an increase in baseline IL-1α in SQ20B cells (FIG. 4H). To confirm that the pan-caspase inhibitor was effectively blocking cell death, we performed a clonogenic assay with erlotinib- and Z-VAD-treated cells in SQ20B cells. Caspase inhibition significantly blocked erlotinib-induced cell death (FIG. 4I) altogether suggesting that IL-1α release may be due to erlotinib-induced cell death.

IL-1R is Negatively Correlated with Survival in HNSCC

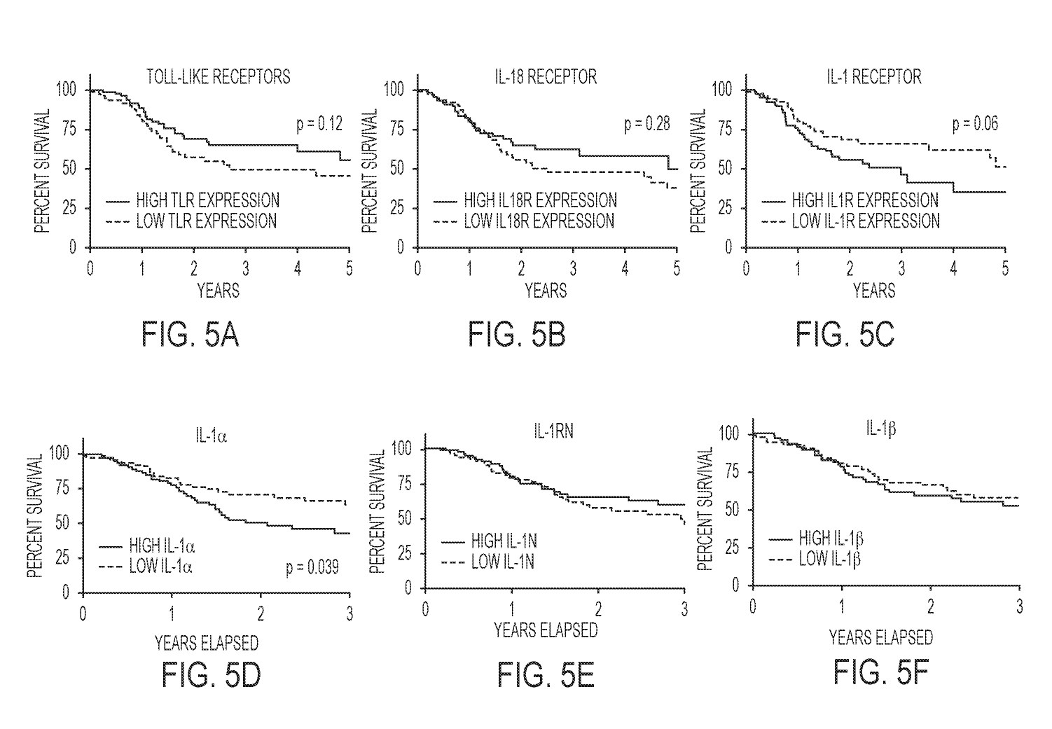

Sequenced HNSCC tumors (n=467) were analyzed from The Cancer Genome Atlas (TCGA). We used this HNSCC dataset to examine the survival of patients with tumors expressing high levels of MyD88-dependent receptors. HNSCC tumors with high expression of TLRs (TLRs 1-10), IL-1R, and IL-18R were plotted for survival against low expressing tumors (FIG. 5). TLRs and IL-18R were not significantly correlated with survival (FIG. 5A,B). However, high IL-1R expressing tumors showed a trend (p=0.06) toward a negative correlation with survival (FIG. 5C). Furthermore, high IL-1R expressing tumors had a median survival time of approximately 3 years, compared to 5 years for low IL-1R expressing tumors (FIG. 5). Survival of HNSCC patients with tumors expressing high and low levels of IL-1 ligands (IL-1α, IL-β and IL-1Ra/IL-1RN) were also examined. IL-1α mRNA was negatively correlated with survival (FIG. 5D), while there was no significant difference in survival for the other ligands (FIG. 5E,F). These data suggest that IL-1α/IL-1R expression may be an important prognostic marker in HNSCC.

Loss of MyD88 Increases Sensitivity to Erlotinib in a Xenograft Model of HNSCC

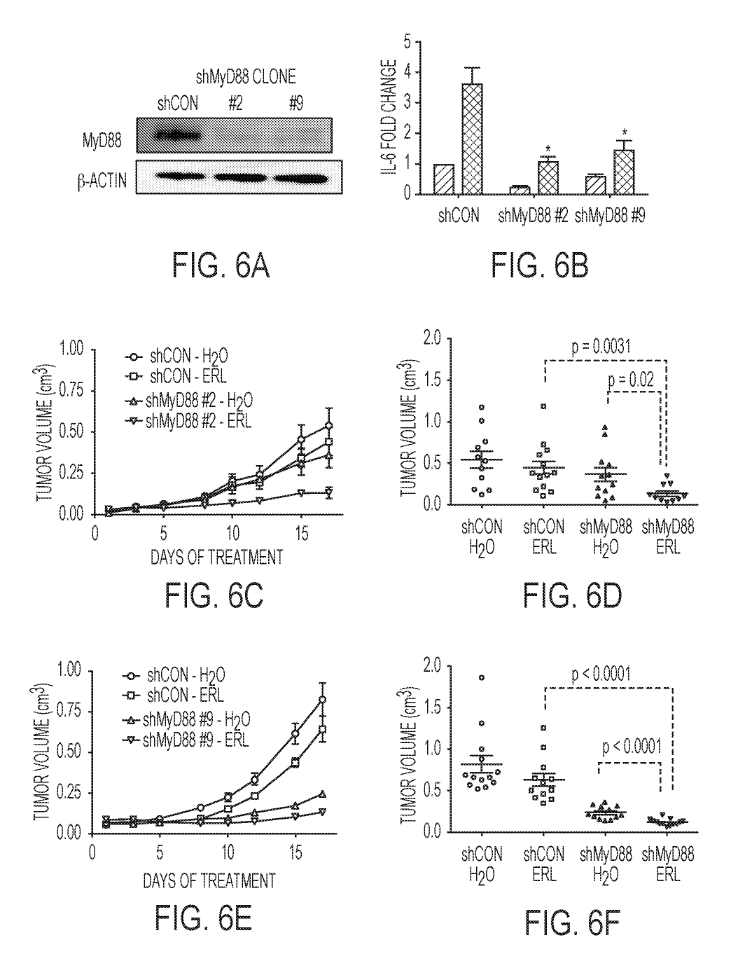

Given that both TLR5 and IL-1R signaling were implicated in erlotinib-induced IL-6 expression, suppression of MyD88 expression was used as a strategy to block all MyD88-dependent signaling. Transient knockdown of MyD88 using siRNA targeted to MyD88 significantly suppressed erlotinib-induced IL-6 production in both Cal-27 and SQ20B cells. SQ20B cells were further chosen for the stable MyD88 knockdown (using shRNA) experiments because of superior transfection efficiency in this cell line and effective knockdown of MyD88 expression (FIG. 6A) compared to our other HNSCC cell lines. After 48-hour treatment with erlotinib, IL-6 secretion was decreased in cells lacking MyD88 expression compared to erlotinib-treated control cells (FIG. 6B) supporting the role of MyD88-dependent signaling in erlotinib-induced IL-6 production.

The above described SQ20B control and MyD88 stable knockout clones were grown as xenografts in nude mice and treated daily with water or erlotinib as described in the Methods section. Both MyD88-deficient xenografts (shMyD88 #2 and #9) showed reduced tumor growth when treated with erlotinib compared to erlotinib-treated control xenografts (FIG. 6C-F). Tumor growth is reported through Day 17 of treatment, as mice in the control group had to be euthanized on Day 17 due to the size of the tumors (FIG. 6D,E). Notably, xenografts bearing the shMyD88 #9 clone showed reduced tumor growth in both treated and untreated groups (FIG. 6E,F). Altogether we show that the IL-1α/IL-1R/MYD88/IL-6 pathway may be responsible for the reduced anti-tumor efficacy of erlotinib and blockade of the MyD88-dependent signaling pathway may improve the efficacy of erlotinib and other EGFRIs in the treatment of HNSCC.

Discussion

Our lab has previously shown that erlotinib and other EGFRIs (lapatinib, cetuximab, panitumumab) increased IL-6 expression and secretion and that increased IL-6 levels played a critical role in erlotinib resistance in vitro and in vivo (10). The studies presented here indicate that MyD88-dependent signaling is most likely responsible for the IL-6 production induced by EGFRIs and resistance to EGFRIs. Therefore targeting MyD88 or receptors that depend on MyD88 for their activity (such as TLRs, IL-1R or IL-18R) may increase the anti-tumor efficacy of erlotinib in HNSCC cells.

Gene expression analyses implicated TLR/MyD88 signaling (especially TLR2/MyD88) as a possible upstream mediator of IL-6 production after erlotinib treatment (FIG. 1D) however we found no evidence of TLR2 involvement despite TLR2 being present and active on HNSCC tumors and cell lines (FIG. 2C-G). Interestingly, TLR5 was active in SQ20B cells (FIG. 2C) and TLR5 knockdown partially but significantly suppressed erlotinib-induced IL-6 production in this cell line only (FIG. 2H). TLR5 has been shown to be a predictive marker for tumor recurrence for tongue squamous cell carcinoma (18) but contrarily, TLR5 may correlate with better prognosis in non-small cell lung cancer (19). Despite these conflicting results, studies have consistently demonstrated that TLR5 expression has radioprotective activity and that radioresistant cells have increased TLR5 expression (20-22). In support of these findings, the SQ20B cell line is a well-documented radioresistant cell line compared to other HNSCC cell lines (23) and was the only cell line to demonstrate TLR5 activity. These findings are currently being pursued in other studies.

The IL-18R and IL-1R both require MyD88 for their downstream activity and IL-6 production (15). While RNA expression of both of these receptors were increased in HNSCC tumor tissue compared to normal matched tissue (FIG. 3A,B), only the IL-1R was found to be involved in erlotinib-induced IL-6 production in both cell lines (FIG. 3C-I) suggesting that the IL-1 pathway may be more involved in poor anti-tumor response to erlotinib compared to TLRs or IL-18R. The IL-1 family includes the ligands IL-1α, IL-1β, and IL-1 receptor antagonist (IL-1Ra) which bind to interleukin-1 receptor types I and II (IL-1R1 and IL-1R2) (24). Of the ligands in the IL-1 family, IL-1β is the most well-studied and its production is dependent on inflammasome-mediated caspase-1 activity (25). In the present studies we believe that IL-1α and not IL-1β is involved in the activation of the IL-1R/MyD88/IL-6 pathway by erlotinib since we were unable to detect any secreted IL-1β by ELISA after erlotinib treatment and neutralization of IL-1β activity did not affect erlotinib-induced IL-6 (FIG. 4E,F). On the other hand, we were able to detect measureable levels of IL-1α by ELISA (FIG. 4A,B) and suppression of IL-1α significantly blocked erlotinib-induced IL-6 (FIG. 4E,F) suggesting that IL-1α was the ligand responsible for activating the IL-1 pathway.

The cytokine IL-1α has a far different biological profile than IL-1β. Unlike IL-1β, IL-1α is not secreted from the cell, but is released during cell death (26). It is likely that the cell death induced by erlotinib treatment resulted in IL-1α release since the use of a pan-caspase inhibitor blocked erlotinib-induced cell death (FIG. 4I) and IL-1α release (FIG. 4H). The IL-1 family ligand IL-1Ra does not induce downstream signaling from IL-1R1, and therefore inhibits the IL-1 pathway through competition with IL-1α and IL-1β for receptor sites (24). In support of this, the use of a humanized recombinant IL-1Ra (Anakinra) effectively blocked erlotinib-induced IL-6 (FIG. 3E,F) suggesting a potential role for anakinra in the treatment of HNSCC in combination with EGFR inhibitors.

We found that HNSCC tumors expressed high levels of IL-1α compared to normal tissue (FIG. 3A,B) and high-IL-1α-expressing tumors have worse prognosis than low-IL-la-expressing tumors (FIGS. 5D). Variable levels of IL-1α expression may explain why some patients respond initially to EGFRI treatment, while others are intrinsically resistant. If IL-1α were truly involved in the limited efficacy of EGFRIs, then patients with tumors already expressing high levels of IL-1α would not respond, or exhibit a lesser response, to EGFR inhibition. Patients with low IL-1α in their tumors would respond initially, and but would become refractory once IL-1α was upregulated by EGFRIs. Unfortunately, since the only large HNSCC dataset with gene expression data (TCGA) did not segregate patients by whether they received EGFRIs, and we were unable to analyze this theory.

Stable knockdown MyD88 clones were generated and used in vivo as a strategy to block all potential signaling from MyD88-dependent receptors. As expected, suppression of MyD88 effectively blocked erlotinib-induced IL-6 production (FIG. 6A,B). Moreover tumor growth was dramatically inhibited in MyD88-deficienct xenografts treated with erlotinib (FIG. 6C-F) suggesting that MyD88 inhibition may be a promising strategy to increase the effect of erlotinib. There are a variety of MyD88 inhibitors currently being developed, but none have been approved to date. These include small molecule inhibitors and inhibitory peptides that prevent dimerization of MyD88 or recruitment of MyD88 to dependent receptors (27-29). Another option is targeting downstream effectors of MyD88 signaling, the most developed of which is an inhibitor of interleukin receptor-associated kinase 4 (IRAK4). A small molecule inhibitor of the kinase activity of IRAK4 has shown initial clinical promise (30). It should be noted, however, that global inhibition of MyD88 may have unexpected results. Our model takes into account only the activity of MyD88 within cancer cells. Inhibition of MyD88 in innate immune cells would change the inflammatory microenvironment especially in an immune competent mouse model, conceivably altering recruitment of immune cells and unpredictably altering growth of the tumor. This remains to be studied.

Based on these findings and our prior studies, we propose a model in which EGFR inhibition causes cell death and release of IL-1α which binds its receptor IL-1R, activates MyD88 and induces IL-6 secretion via NF-κB. IL-6 signaling pathways lead to phosphorylation of STAT3, which is well known to compensating for the loss of EGFR signaling due to cross talk (31). As such, we believe that the poor response to the EGFR inhibitor erlotinib in the clinical setting may be due to IL-1α inducing IL-6 production through MyD88-dependent pathways. To our knowledge, the studies presented here are the first to connect MyD88-dependent signaling with resistance to EGFR-targeted therapy. This novel mechanism offers insight into why other methods of overcoming EGFRI resistance have failed, and proposes new clinical targets that may enhance the efficacy of EGFRIs in HNSCC.

### EXAMPLE 1

### References

1. Loeffler-Ragg J, Schwentner I, Sprinzl G M, Zwierzina H. EGFR inhibition as a therapy for head and neck squamous cell carcinoma. Expert Opin Investig Drugs 2008;17:1517-31.
2. Bonner J A, Harari P M, Giralt J, Azarnia N, Shin D M, Cohen R B, et al. Radiotherapy plus cetuximab for squamous-cell carcinoma of the head and neck. N Engl J Med 2006;354:567-78.
3. Cohen E E, Kane M A, List M A, Brockstein B E, Mehrotra B, Huo D, et al. Phase II trial of gefitinib 250 mg daily in patients with recurrent and/or metastatic squamous cell carcinoma of the head and neck. Clin Cancer Res 2005;11:8418-24.
4. Soulieres D, Senzer N N, Vokes E E, Hidalgo M, Agarwala S S, Siu L L. Multicenter phase II study of erlotinib, an oral epidermal growth factor receptor tyrosine kinase inhibitor, in patients with recurrent or metastatic squamous cell cancer of the head and neck. J Clin Oncol 2004;22:77-85.
5. Vermorken J B, Trigo J, Hitt R, Koralewski P, Diaz-Rubio E, Rolland F, et al. Open-label, uncontrolled, multicenter phase II study to evaluate the efficacy and toxicity of cetuximab as a single agent in patients with recurrent and/or metastatic squamous cell carcinoma of the head and neck who failed to respond to platinum-based therapy. J Clin Oncol 2007;25:2171-7.
6. Argiris A, Kotsakis A P, Hoang T, Worden F P, Savvides P, Gibson M K, et al. Cetuximab and bevacizumab: preclinical data and phase II trial in recurrent or metastatic squamous cell carcinoma of the head and neck. Ann Oncol 2013;24:220-5.
7. de Souza J A, Davis D W, Zhang Y, Khattri A, Seiwert T Y, Aktolga S, et al. A phase II study of lapatinib in recurrent/metastatic squamous cell carcinoma of the head and neck. Clin Cancer Res 2012;18:2336-43.
8. Miller V A, Hirsh V, Cadranel J, Chen Y M, Park K, Kim S W, et al. Afatinib versus placebo for patients with advanced, metastatic non-small-cell lung cancer after failure of erlotinib, gefitinib, or both, and one or two lines of chemotherapy (LUX-Lung 1): a phase 2b/3 randomised trial. Lancet Oncol 2012;13:528-38.
9. Ross H J, Blumenschein G R, Jr., Aisner J, Damjanov N, Dowlati A, Garst J, et al. Randomized phase II multicenter trial of two schedules of lapatinib as first- or second-line monotherapy in patients with advanced or metastatic non-small cell lung cancer. Clin Cancer Res 2010;16:1938-49.
10. Fletcher E V, Love-Homan L, Sobhakumari A, Feddersen C R, Koch A T, Goel A, et al. EGFR inhibition induces proinflammatory cytokines via NOX4 in HNSCC. Mol Cancer Res 2013;11:1574-84.
11. Duffy S A, Taylor J M, Terrell J E, Islam M, Li Y, Fowler K E, et al. Interleukin-6 predicts recurrence and survival among head and neck cancer patients. Cancer 2008;113:750-7.
12. Heimdal J H, Kross K, Klementsen B, Olofsson J, Aarstad H J. Stimulated monocyte IL-6 secretion predicts survival of patients with head and neck squamous cell carcinoma. BMC Cancer 2008;8:34.
13. Kamimura D, Ishihara K, Hirano T. IL-6 signal transduction and its physiological roles: the signal orchestration model. Rev Physiol Biochem Pharmacol 2003;149:1-38.
14. Riedel F, Zaiss I, Herzog D, Gotte K, Naim R, Hormann K. Serum levels of interleukin-6 in patients with primary head and neck squamous cell carcinoma. Anticancer Res 2005;25:2761-5.
15. Martin M U, Wesche H. Summary and comparison of the signaling mechanisms of the Toll/interleukin-1 receptor family. Biochim Biophys Acta 2002;1592:265-80.
16. Weichselbaum R R, Dahlberg W, Beckett M, Karrison T, Miller D, Clark J, et al. Radiation-resistant and repair-proficient human tumor cells may be associated with radiotherapy failure in head- and neck-cancer patients. Proc Natl Acad Sci USA 1986;83:2684-8.
17. Spitz D R, Malcolm R R, Roberts R J. Cytotoxicity and metabolism of 4-hydroxy-2-nonenal and 2-nonenal in H202-resistant cell lines. Do aldehydic by-products of lipid peroxidation contribute to oxidative stress? Biochem J 1990;267:453-9.
18. Kauppila J H, Mattila A E, Karttunen T J, Salo T. Toll-like receptor 5 (TLRS) expression is a novel predictive marker for recurrence and survival in squamous cell carcinoma of the tongue. Br J Cancer 2013;108:638-43.
19. Zhou H, Chen J H, Hu J, Luo Y Z, Li F, Xiao L, et al. High expression of Toll-like receptor 5 correlates with better prognosis in non-small-cell lung cancer: an anti-tumor effect of TLR5 signaling in non-small cell lung cancer. J Cancer Res Clin Oncol 2014;140:633-43.
20. Wang Z D, Qiao YL, Tian X F, Zhang X Q, Zhou S X, Liu H X, et al. Toll-like receptor 5 agonism protects mice from radiation pneumonitis and pulmonary fibrosis. Asian Pac J Cancer Prey 2012;13:4763-7.
21. Burdelya LG, Gleiberman A S, Toshkov I, Aygun-Sunar S, Bapardekar M, Manderscheid-Kern P, et al. Toll-like receptor 5 agonist protects mice from dermatitis and oral mucositis caused by local radiation: implications for head-and-neck cancer radiotherapy. Int J Radiat Oncol Biol Phys 2012;83:228-34.
22. Roses R E, Xu M, Koski G K, Czerniecki B J. Radiation therapy and Toll-like receptor signaling: implications for the treatment of cancer. Oncogene 2008;27:200-7.
23. Gupta A K, McKenna W G, Weber C N, Feldman M D, Goldsmith J D, Mick R, et al. Local recurrence in head and neck cancer: relationship to radiation resistance and signal transduction. Clin Cancer Res 2002;8:885-92.
24. Boraschi D, Tagliabue A. The interleukin-1 receptor family. Semin Immunol 2013;25:394-407.

25. Bergsbaken T, Fink S L, Cookson B T. Pyroptosis: host cell death and inflammation. Nat Rev Microbiol 2009;7:99-109.

26. Kim B, Lee Y, Kim E, Kwak A, Ryoo S, Bae SH, et al. The Interleukin-lalpha Precursor is Biologically Active and is Likely a Key Alarmin in the IL-1 Family of Cytokines. Front Immunol 2013;4:391.

27. Bartfai T, Behrens M M, Gaidarova S, Pemberton J, Shivanyuk A, Rebek J, Jr. A low molecular weight mimic of the Toll/IL-1 receptor/resistance domain inhibits IL-1 receptor-mediated responses. Proc Natl Acad Sci USA 2003;100:7971-6.

28. Loiarro M, Volpe E, Ruggiero V, Gallo G, Furlan R, Maiorino C, et al. Mutational analysis identifies residues crucial for homodimerization of myeloid differentiation factor 88 (MyD88) and for its function in immune cells. J Biol Chem 2013;288:30210-22.

29. Loiarro M, Sette C, Gallo G, Ciacci A, Fanto N, Mastroianni D, et al. Peptide-mediated interference of TIR domain dimerization in MyD88 inhibits interleukin-1-dependent activation of NF-{kappa} B. J Biol Chem 2005;280:15809-14.
30. Tumey L N, Boschelli D H, Bhagirath N, Shim J, Murphy E A, Goodwin D, et al. Identification and optimization of indolo[2,3-c]quinoline inhibitors of IRAK4. Bioorg Med Chem Lett 2014;24:2066-72.
31. Colomiere M, Ward A C, Riley C, Trenerry M K, Cameron-Smith D, Findlay J, et al. Cross talk of signals between EGFR and IL-6R through JAK2/STAT3 mediate epithelial-mesenchymal transition in ovarian carcinomas. Br J Cancer 2009;100:134-44.

### EXAMPLE 2

Abstract

Epidermal growth factor receptor (EGFR) is upregulated in the majority of head and neck squamous cell carcinomas (HNSCC). However many HNSCC patients respond poorly to the EGFR inhibitors (EGFRIs) cetuximab and erlotinib despite tumor expression of EGFR. Gene expression analysis of erlotinib-treated HNSCC cells revealed an upregulation of genes involved in MyD88-dependent signaling compared to their respective vehicle-treated cell lines. We therefore investigated if MyD88-dependent signaling may reduce the anti-tumor efficacy of EGFRIs in HNSCC. Erlotinib significantly upregulated interleukin-6 (IL-6) secretion in HNSCC cell lines which our laboratory previously reported to result in reduced drug efficacy. Suppression of MyD88 expression blocked erlotinib-induced IL-6 secretion in vitro and increased the anti-tumor activity of erlotinib in vivo. There was little evidence of toll-like receptor or interleukin-18 receptor involvement in erlotinib-induced IL-6 secretion. However, suppression of interleukin-1 receptor (IL-1R) signaling significantly reduced erlotinib-induced IL-6 production. A time-dependent increase of IL-1 alpha (IL-1α) but not IL-1 beta (IL-1β) was observed in response to erlotinib treatment and IL-1α blockade significantly increased the anti-tumor activity of erlotinib and cetuximab in vivo. A pan-caspase inhibitor reduced erlotinib-induced IL-1α secretion suggesting that IL-1α was released due to cell death. Human HNSCC tumors showed higher IL-1α mRNA levels compared to matched normal tissue, and IL-1α was found to be negatively correlated with survival in HNSCC patients. Overall, the IL-1α/IL-1R/MYD88/IL-6 pathway may be responsible for the reduced anti-tumor efficacy of erlotinib and other EGFRIs; and blockade of IL-1 signaling may improve the efficacy of EGFRIs in the treatment of HNSCC.

Introduction

The epidermal growth factor receptor (EGFR) is a receptor tyrosine kinase that activates numerous pro-survival pathways including Akt and STAT3 signaling pathways (1). Given that EGFR signaling is upregulated in many cancers especially head and neck squamous cell carcinoma (HNSCC), several drugs that target EGFR have been developed and approved for cancer therapy such as monoclonal antibodies that block the extracellular ligand binding domain (e.g. cetuximab, panitumumab) and small molecule tyrosine kinase inhibitors (TKIs) that prevent activation of the cytoplasmic tyrosine kinase domain (e.g. gefitinib, erlotinib) (1). To date, only cetuximab is FDA approved for use in HNSCC, however it should be noted that response rates to cetuximab as a single agent are quite low (13%) and of limited duration (2-3 months). Similarly, low response rates (4-11%) have been observed in clinical trials with HNSCC patients treated with gefitinib and erlotinib (2-5). Many different mechanisms (e.g. existing/acquired mutations and alternative signaling pathways) have been proposed that may reduce patient response to EGFRIs, but this knowledge has not improved survival rates for HNSCC patients to date (6-9).

Previous studies in our laboratory observed a significant upregulation in IL-6 expression in HNSCC cell lines treated with EGFRIs (10). IL-6 is a pleotropic cytokine with a wide range of biological activities and is well known for its role in inflammation, tumor progression and chemoresistance in HNSCC (11-14). We additionally demonstrated the ability of IL-6 signaling to protect HNSCC against erlotinib (ERL) treatment in vitro and in vivo (10) supporting prior reports showing that IL-6 may be involved in resistance to EGFRIs (15-18).

A well-established mechanism of IL-6 production involves the cytosolic adaptor protein myeloid differentiation primary response gene 88 (MyD88), which acts through intermediaries to induce nuclear factor kappa-light-chain-enhancer of activated B cells (NFκB) activation (19). MyD88 is required for the activity of members of the Toll/Interleukin-1 receptor (TIR) superfamily which include Toll-like Receptors (TLRs), the Interleukin-1 Receptor (IL-1R), and the IL-18 Receptor (IL-18R) (19). Activation of these receptors lead to the recruitment of MyD88 via its TIR domain resulting in NFkB activation and expression of pro-inflammatory cytokines including IL-6 (19). Here we show that EGFR inhibition using ERL activates the IL-1α/IL-1R/MyD88/IL-6 signaling pathway and this pathway may serve as a novel mechanism responsible for the poor long-term anti-tumor efficacy of EGFRIs in HNSCC therapy.

Materials and Methods

Cells and Culture Conditions:

Cal-27 and FaDu human head and neck squamous carcinoma (HNSCC) cells were obtained from the American Type Culture Collection (ATCC, Manassas, Va.). SQ20B HNSCC cells (20) were a gift from Dr. Anjali Gupta (Department of Radiation Oncology, The University of Iowa). All HNSCC cell lines are EGFR positive and are sensitive to EGFR inhibitors. All cell lines were authenticated by the ATCC for viability (before freezing and after thawing), growth, morphology and isoenzymology. Cells were stored according to the supplier's instructions and used over a course of no more than 3 months after resuscitation of frozen aliquots. Cultures were maintained in 5% CO₂ and air humidified in a 37° C. incubator.

In Vitro Drug Treatment:

Erlotinib (ERL; Tarceva), anakinra (ANA; Kineret) and N-acetyl cysteine (NAC; Acetadote) were obtained from the inpatient pharmacy at the University of Iowa Hospitals and Clinics. Drugs were added to cells at final concentrations of 5 μM ERL, 10 ng/mL or 50 ng/mL ANA and 20 mM NAC. Human IgG and dimethyl sulfoximine (DMSO) were used as controls and were obtained from Sigma Aldrich. Pegylated catalase (CAT; Sigma Aldrich) was used at a final concentration of 100 U/mL. Human IL-1α, IL-1β, and IL-18Ra neutralizing antibodies were obtained from R&D Systems and were used at a concentration of 0.5 μg/mL. Recombinant human IL-1α was obtained from Life Technologies and administered at a concentration of 1 ng/mL. Ac-Y-VAD-cho (CalBioChem) was suspended in DMSO and used at 5 μM. Z-VAD-fmk (Promega) was diluted in DMSO and used at 20 μM. TLR agonists were used at the following concentrations: Pam3CSK4 (200 ng/mL), FSL-1 (100 ng/mL), Poly I:C (20 μg/mL), LPS (200 ng/mL), Flagellin (200 ng/mL), Gardiquimod (1 μg/mL), CL075 (1 μg/mL), and E. coli DNA (1 μg/mL). All TLR agonists were obtained from InvivoGen. The required volume of each drug was added directly to complete cell culture media on cells to achieve the indicated final concentrations.

Microarray Analyses:

Gene expression analysis of HNSCC cells treated with DMSO or erlotinib (5 μM, 48 h) has been described previously (GeneBank accession no. GSE45891 (10)). Downstream pathway, network, process and disease analyses of the resultant gene expression data for all cell lines (n=3 experiments per cell line) was carried out using

MetacoreTM (GeneGo) using a threshold of +1.3 and a p-value of 0.05. Enrichment analysis of the resultant gene expression profiles of SQ20B and Cal-27 HNSCC cells exposed to ERL versus DMSO was performed by mapping gene IDs from the resultant dataset onto gene IDs in built-in functional ontologies which include cellular/molecular process networks, disease biomarker networks, canonical pathway maps and metabolic networks.

Real-Time Quantitative PCR:

Total RNA was extracted from cells after indicated time points using RNeasy.Plus mini kit (Qiagen). Conversion of RNA into cDNA was accomplished with the iScript cDNA synthesis kit (Bio-Rad) and a thermocycler with the following conditions: 5 minutes at 25° C., 30 minutes at 42° C., and 5 minutes at 85° C. Subsequent RTPCR analysis was performed in a 96-well optical plate with each well containing 6 μL of cDNA, 7.5 μL of SyBr Green Universal SuperMix (Bio-Rad), and 1.5 μof oligonucleotide primers (sense and antisense; 4 μM) for a total reaction volume of 15 μL. Oligonucleotide primers for human genes were obtained from IDT (Iowa City, Iowa). RTPCR was performed on ABI PRISM Sequence Detection System (model 7000, Applied Biosystems) with the following protocol: 95° C. for 15 seconds (denaturing) and 60° C. for 60 seconds (annealing), repeated for 40 cycles. Threshold cycle (CT) values for analyzed genes (in duplicate) were normalized as compared to GAPDH (cell lines) or 18S (human samples) CT values. Relative abundance was calculated as 0.5̂(ΔCT), with OCT being the CT value of the analyzed gene minus the CT value of the reference gene (GAPDH or 18S).

Western Blot Analysis:

Cell lysates were standardized for protein content, resolved on 4% -12% SDS polyacrylamide gels, and blotted onto nitrocellulose membranes. Membranes were probed with rabbit anti-MyD88 (1:500, Cell Signaling), anti-IL-1R1 (1:500, Santa Cruz), anti-beta-actin (1:5000, Thermo Scientific). Antibody binding was detected by using an ECL Chemiluminescence Kit (Amersham).

Enzyme-Linked Immunosorbent Assay:

Levels of IL-6, IL-1α and IL-1β of treated cells were determined by ELISA. The culture media of the treated cells were harvested and each cytokine was detected according to the manufacturer's protocol using Human Quantikine ELISA Kits (R&D Systems, Minneapolis, Minn.).

Adenoviral Vectors:

Construction and characterization of adenoviral vectors encoding wild-type and dominant negative NADPH oxidase-4 (NOX4) have each been described previously (10, 21). An empty vector lacking the NOX4 construct was used as a control. All vectors were obtained from the University of Iowa Gene Vector Core. HNSCC cells in serum free media were infected with 100 MOI of the above described adenoviral vectors for 24 hours. Biochemical analyses were performed 72-96 h after transfection.

siRNA/shRNA Transfection:

MyD88, TLR2, TLRS and control siRNA (Santa Cruz) were transfected into HNSCC cells at a concentration of 40-80 nM with equal volume Lipofectamine RNAiMAX (Invitrogen). Cells were incubated in Opti-MEM for 4 hours prior to addition of siRNA and 16 hours after addition of siRNA. For shRNA transfection, SQ20B cells were transfected with 1 μg/mL of psiRNA-h7SKGFPzeo, psiRNA-shMyD88, or psiRNA-shIL1R (Invivogen) in the presence of Opti-MEM and Lipofectamine RNAiMAX. Cells were allowed to recover 48-72 hours in antibiotic-free DMEM with 10% FBS before 48-hour erlotinib treatment. Knockdown was confirmed by RT-PCR and/or western blot.

Clonogenic Survival Assay:

Clonogenic survival was determined as previously described (22). Individual assays were performed with multiple dilutions with at least four cloning dishes per data point, repeated in at least 3 separate experiments.

Tumor Cell Implantation:

Male and female athymic-nu/nu mice (4-5 weeks old) were purchased from Harlan Laboratories (Indianapolis, Ind.). Mice were housed in a pathogen-free barrier room in the Animal Care Facility at the University of Iowa and handled using aseptic procedures. All procedures were approved by the IACUC committee of the University of Iowa and conformed to the guidelines established by the NIH. Mice were allowed at least 3 days to acclimate prior to beginning experimentation, and food and water were made freely available. Tumor cells were inoculated into nude mice by subcutaneous injection of 0.1 mL aliquots of saline containing 2×10⁶ SQ20B cells into the right flank using 26-gauge needles.

In Vivo Drugs Administration:

Mice started drug treatment 1 week after tumor inoculation. For the MyD88 knockdown experiments, female mice were randomized into 2 treatment groups and orally administered either water or 12.5 mg/kg erlotinib (ERL) daily. For the IL-1α neutralization experiments, male and female mice were randomized into 4 treatment groups as follows. Control group: Mice were administered water orally daily and 1 mg/kg IgG i.p once per week. Neutralizing IL-1α antibody (nIL-1 aab) group: For experiments involving cetuximab (CTX), CTX was administered i.p. 2 mg per mouse twice per week and control mice were given IgG twice per week. All treatments were given for the duration of three weeks. Mice were evaluated daily and tumor measurements taken three times per week using Vernier calipers. Tumor volumes were calculated using the formula: tumor volume=(length×width²)/2 where the length was the longest dimension, and width was the dimension perpendicular to length. Mice were euthanized via CO₂ gas asphyxiation or lethal overdose of sodium pentobarbital (100 mg/kg) when tumor diameter exceeded 1.5 cm in any dimension.

Bioinformatics:

The Cancer Genome Browser (University of California-Santa Cruz; https://genome-cancer.ucsc.edu) was used to download the level 3 dataset HNSCC dataset (TCGA_HNSC_exp_HiSeqV2_PANCAN) from The Cancer Genome Atlas (TCGA). RNAseq data was normalized across all TCGA cohorts and reported as log2 values. Corresponding level 3 clinical data was available for most of the 467 samples. Selected tumors (n=41) also had RNAseq data for matched normal tissue. Matched tumor and normal samples were analyzed. Linear fold change was calculated to emphasize difference between groups. Kaplan-Meier survival curves were generated by comparing survival of the highest quartile of expressing tumors (for indicated gene) against the lowest quartile. In some cases, Kaplan-Meier curves were generated using an aggregate of several genes. The genes aggregated are as follows: TLR (TLR1,TLR2, TLR4,TLR5,TLR6,TLR7,TLR8,TLR9,TLR10), IL-18R (IL 18Ra,IL18Rb), IL-1R survival curve (IL1R1,IL1RAP), IL-1α, IL-1β and IL-1RA/IL-1RN). Tumors were ranked according to expression of each gene, and ranks were averaged to determine highest and lowest quartile of tumors expressing the given receptor family.

Statistical Analysis:

Statistical analysis was done using GraphPad Prism version 5 for Windows (GraphPad Software, San Diego, Calif.). Differences between 3 or more means were determined by one-way ANOVA with Tukey post-tests. Linear mixed effects regression models were used to estimate and compare the group-specific change in tumor growth curves. Differences in survival curves were determined by Mantel-Cox test. All statistical analysis was performed at the p<0.05 level of significance.

Results

Erlotinib Induces Processes Involved in Inflammation

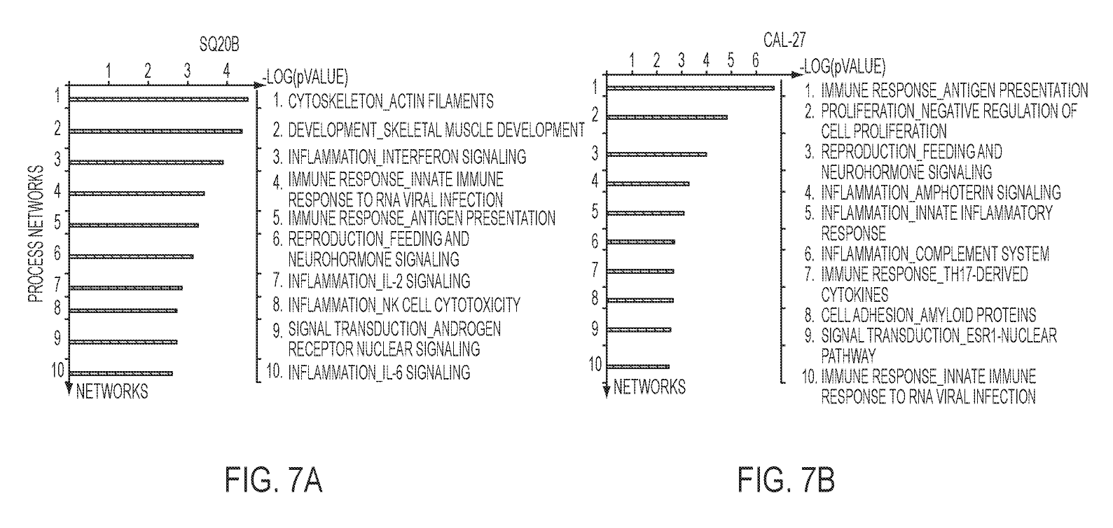

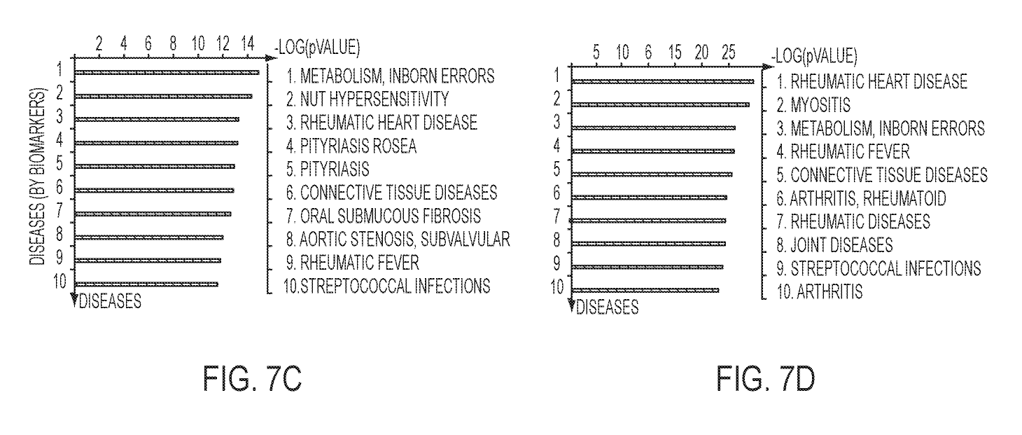

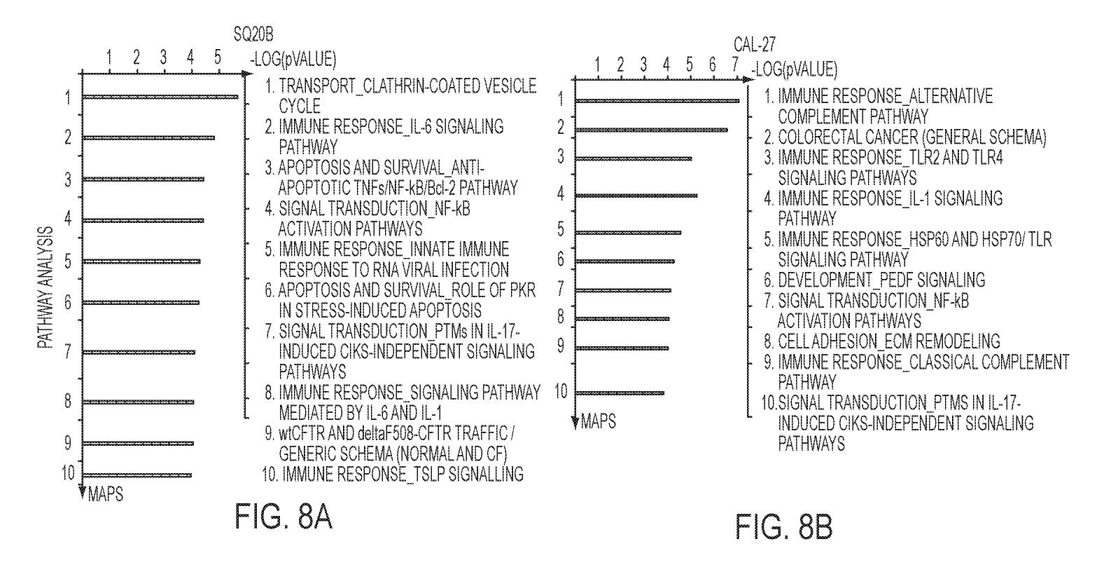

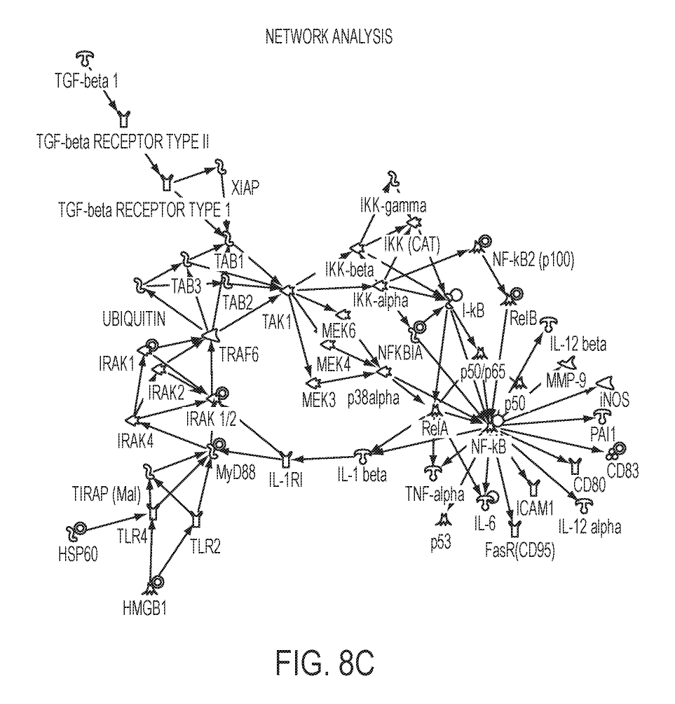

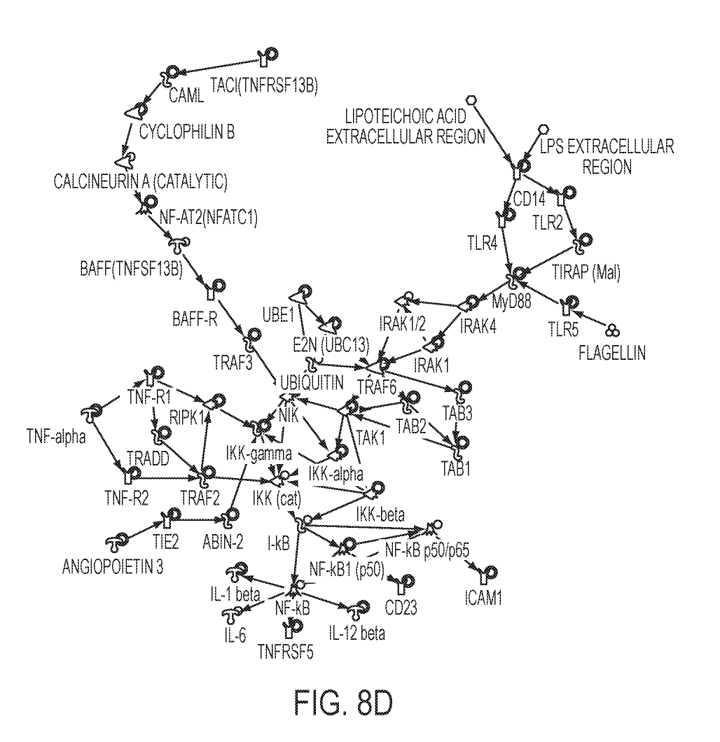

Of the top ten upregulated cellular process networks identified by ERL treatment, 6 processes were related to immune response or inflammation for both cell lines (FIG. 7A,B). The top ten significant diseases that were identified from ERL treatment were predominantly systemic inflammatory disorders in both cell lines such as rheumatic diseases/disorders (rheumatic arthritis, rheumatic fever, rheumatic heart disease) (FIG. 7C,D). Similarly, the majority of the top ten upregulated canonical pathways were immune response/inflammation related in both cell lines which included IL-6 and IL-1 signaling in SQ20B cells (FIG. 2A) and TLR and IL-1 signaling in Cal-27 cells (FIG. 8B).

The top network identified for SQ20B and Cal-27 was the NF-kB, MyD88, I-kB, IRAK1/2, NF-kB2 (p100) network (FIG. 8C) and TRAF6, TAK1(MAP3K7), NF-kB, I-kB, IKK-gamma network (FIG. 8D) respectively. The genes and processes in these networks were both related to MyD88-dependent TLR signaling and NFkB activity. Altogether, the gene expression analyses suggested that ERL activates inflammatory processes and pathways which may be mediated by MyD88.

Loss of MyD88 Increases Tumor Sensitivity to Erlotinib

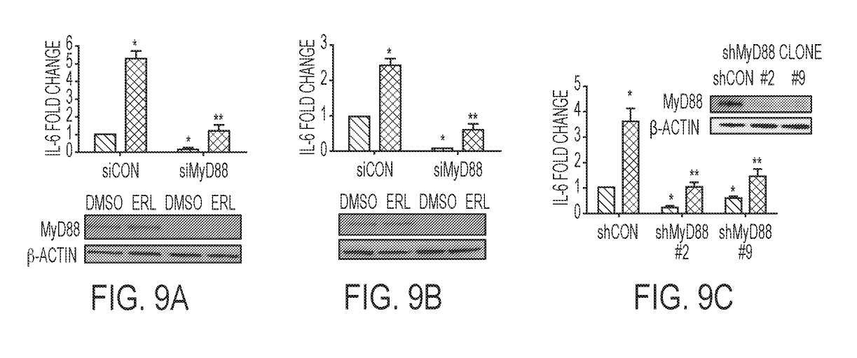

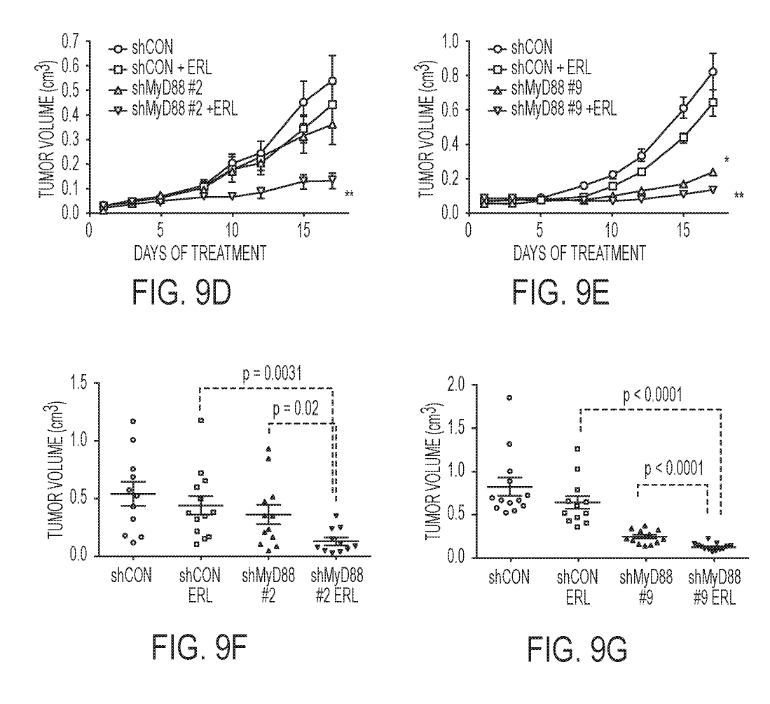

We have previously shown that ERL induces the secretion of IL-6 and other proinflammatory cytokines via NFkB activation in HNSCC cells (10) which supports the gene expression results (FIG. 7,8). Transient knockdown of MyD88 significantly suppressed baseline and ERL-induced IL-6 production in both SQ20B (FIG. 9A) and Cal-27 cells (FIG. 9B). MyD88 stable knockout clones (shMyD88#2, shMyD88#9) also demonstrated significantly reduced IL-6 in the absence and presence of ERL compared to control (FIG. 9C) supporting the role of MyD88-dependent signaling in ERL-induced IL-6 production. Both MyD88 knockout clones showed reduced tumor growth when treated with ERL compared to ERL-treated control xenografts (FIG. 9D-G). Notably, xenografts bearing the shMyD88 #9 clone showed reduced tumor growth in both treated and untreated groups (FIG. 9D,G). Altogether these results suggest that MyD88-dependent signaling is involved in ERL-induced IL-6 secretion and suppresses the anti-tumor activity of ERL.

TLR5 Signaling may be Involved in Erlotinib-Induced IL-6 Secretion

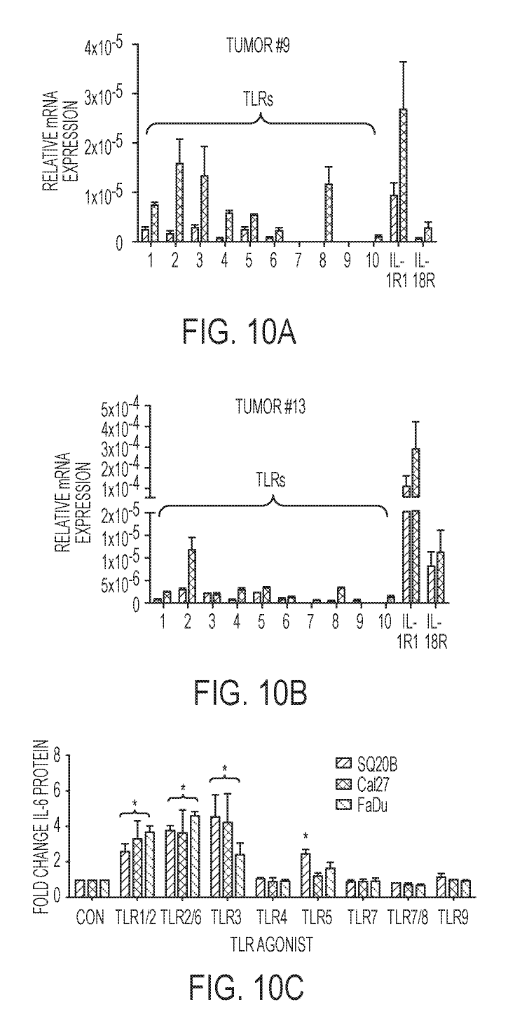

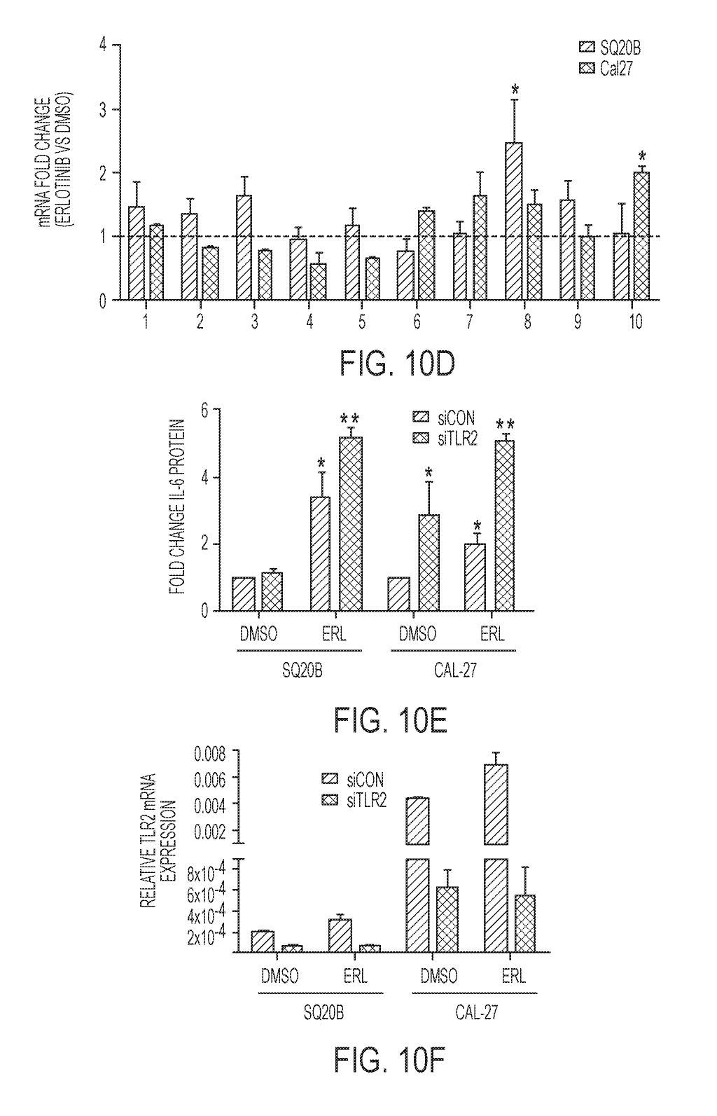

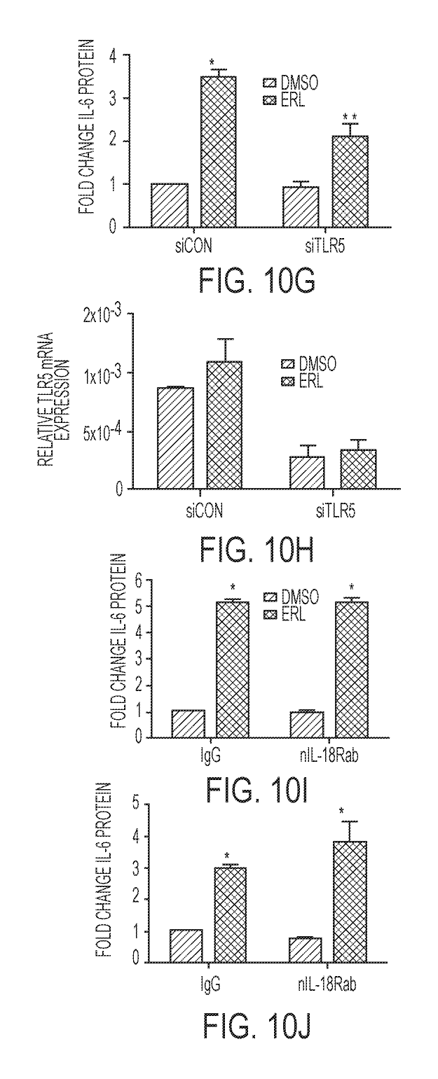

A general trend of increased TLR, IL-1R and IL-18R RNA expression was found in HNSCC human tumors (obtained from the Tissue Procurement Core (TPC) in the Department of Pathology) compared to matched normal tissue (FIG. 10A,B). Notably, both tumors showed large increases in expression of TLR2 compared to normal matched tissue (FIG. 10A,B). IL-6 secretion was significantly increased after treatment with agonists to TLR1/2, TLR2/6 and TLR3 in all 3 cell lines (FIG. 10C), although TLR5 appeared to be active in only SQ20B cells (FIG. 10C). ERL increased TLR8 expression in SQ20B cells and TLR10 in Cal-27 cells although the absolute levels of these TLRs were very low and most likely not of biological significance (FIG. 10D). As the TLR1/2 and TLR2/6 dimers both depend on TLR2, the activity of these dimers were suppressed using siRNA targeted to TLR2 (FIG. 10E,F). Knockdown of TLR2 expression did not decrease ERL-induced IL-6 (FIG. 10E). However, knockdown of TLR5 expression partially but significantly suppressed ERL-induced IL-6 secretion in SQ20B cells (FIG. 10G,H) which was not observed in Cal-27 cells (data not shown). TLR3, which is not a MyD88-dependent receptor also was not involved in ERL-induced IL-6 in both cell lines. Altogether, these results suggest that of the TLRs, only TLR5 signaling may contribute to IL-6 secretion induced by ERL in select HNSCC cell lines.

IL-1 Signaling is Critical for Erlotinib-Induced IL-6 Expression in HNSCC Cells

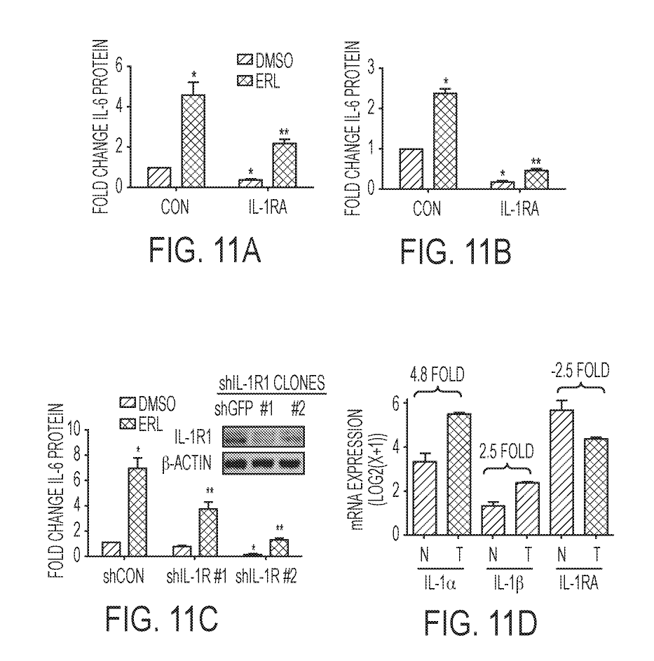

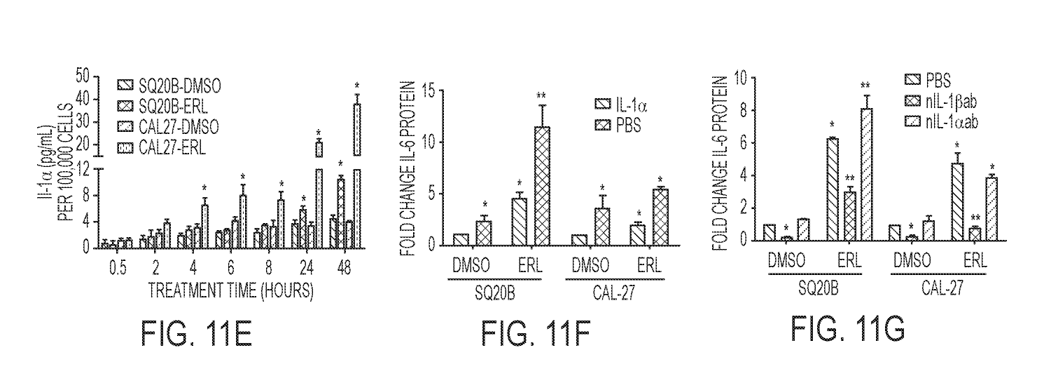

In order to investigate the contribution of other MyD88-dependent signaling pathways, the IL-18R and IL-1R pathways were studied. Neutralization of IL-18R in SQ20B (FIG. 10I) and Cal-27 (FIG. 10J) failed to suppress ERL-induced IL-6. However, anakinra, a recombinant IL-1R antagonist (IL-1RA/IL-1RN) significantly reduced baseline and ERL-induced IL-6 in both SQ20B (FIG. 11A) and Cal-27 (FIG. 11B). Additionally, transient and stable knockdown of the IL-1R suppressed ERL-induced IL-6 (FIG. 11C) suggesting that IL-1R signaling may be involved in ERL-induced IL-6. Sequenced HNSCC tumors and matched normal tissue (n=40) were analyzed from The Cancer Genome Atlas (TCGA) for mRNA levels of ligands of the IL-1 pathway. IL-1α and IL-1β were found to be increased in tumors by 4.8 fold and 2.5 fold respectively compared to normal samples while IL-1RA/IL-1RN was decreased by 2.5 fold (FIG. 11D). IL-1α was also upregulated in both HNSCC tumors analyzed in FIG. 10A,B while IL-1β was only upregulated in one of these tumors. IL-1α but not IL-1β was detectable after ERL treatment and increased across all time points measured in both cell lines (FIG. 11E). Exogenous IL-1α increased IL-6 secretion in the presence and absence of ERL (FIG. 11F) and blockade of IL-1α abut not of IL-1β activity significantly reduced IL-6 secretion in the absence and presence of ERL (FIG. 11G) suggesting that IL-1α release may be responsible for ERL-induced IL-6 production.

Erlotinib-Induced Cell Death Triggers IL-1α Release.

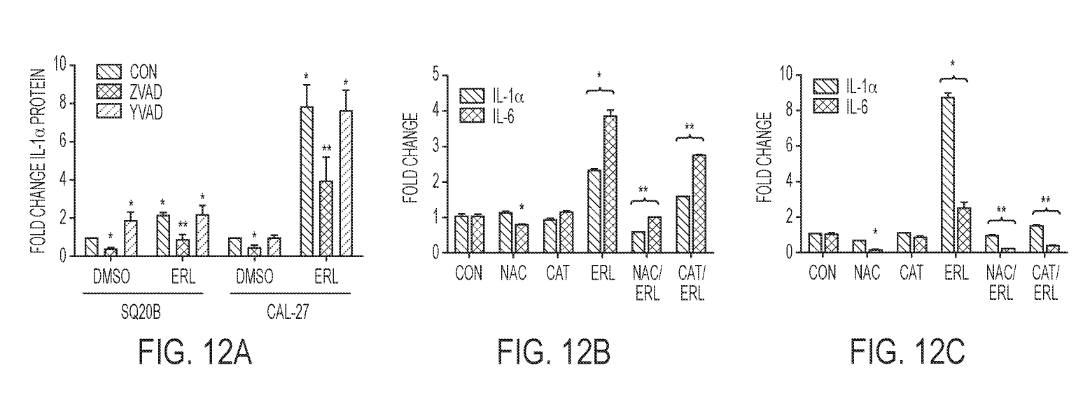

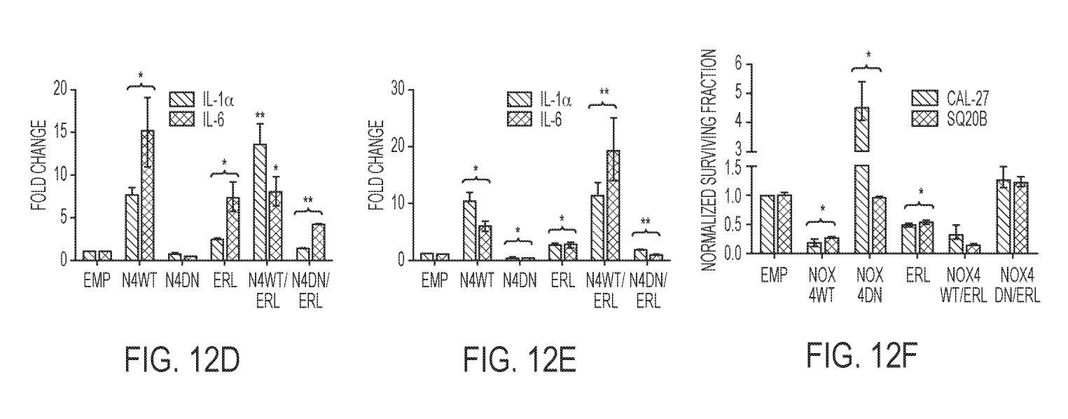

IL-1α unlike IL-1β is not secreted but is typically released by cell death. To confirm this, we showed that Z-VAD-fmk (ZVAD), a pan-caspase inhibitor, significantly reduced baseline and ERL-induced levels of IL-1α (FIG. 12A) and blocked ERL-induced cell death suggesting that IL-1α is likely released due to ERL-induced cell death. These results were not observed with the caspase-1 inhibitor, Ac-Y-VAD-cho (YVAD, FIG. 12A). Our laboratory has previously shown that ERL induces cell death via hydrogen peroxide (H₂O₂)-mediated oxidative stress due to NADPH oxidase-4 (NOX4) activity (23). To confirm that oxidative stress is involved in IL-1α release we showed that the antioxidants NAC and CAT significantly suppressed ERL-induced IL-1α in addition to IL-6 in both SQ20B (FIG. 12B) and Cal-27 cells (FIG. 12C). We have previously shown that these antioxidants significantly protect these HNSCC cell lines from ERL-induced cytotoxicity (23). Moreover, overexpression of dominant negative NOX4 (N4dn) decreased ERL-induced IL-1α, IL-6 production (FIG. 12D,E) and cytotoxicity (FIG. 12F) in both SQ20B (FIG. 12D,F) and Cal-27 (FIG. 12E,F). The opposite results were observed with wildtype NOX4 (N4wt) (FIG. 12D-F). The ability of N4wt (and not N4dn) to significantly induce oxidative stress in these cell lines has been demonstrated in our previous publications (10, 21). Altogether, these results suggest that ERL-induced oxidative stress (via NOX4) results in cell death leading to IL-1α release resulting in activation of IL-1R signaling in unaffected/surviving cells leading to IL-6 expression and secretion.

IL-1α is negatively correlated with survival in HNSCC

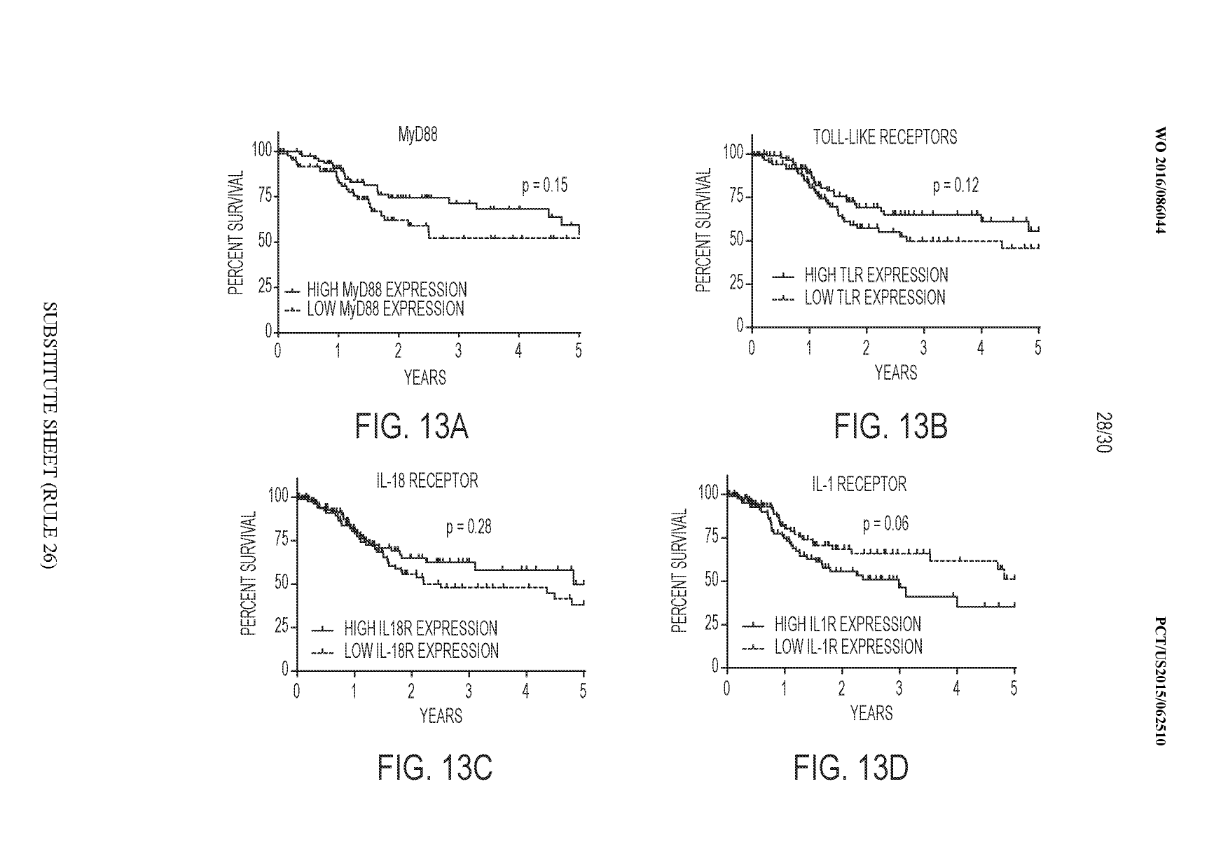

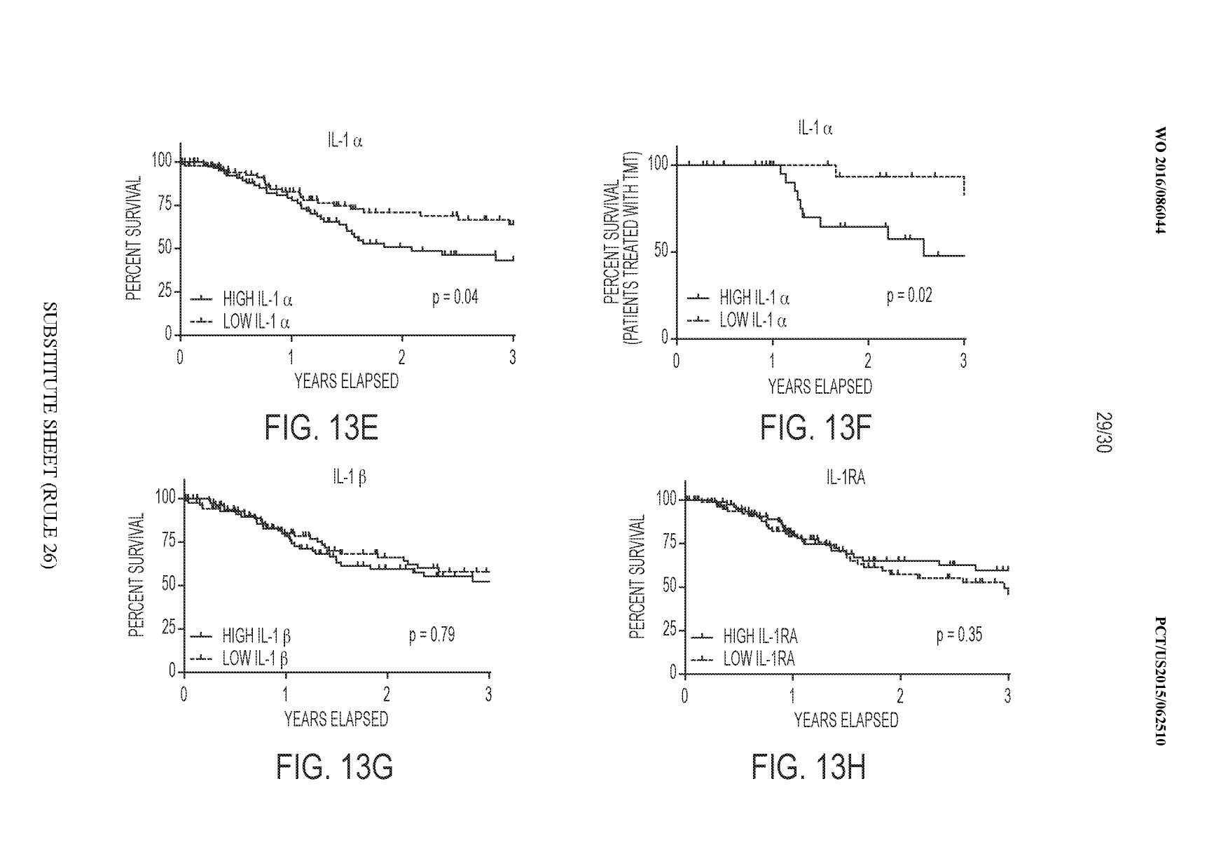

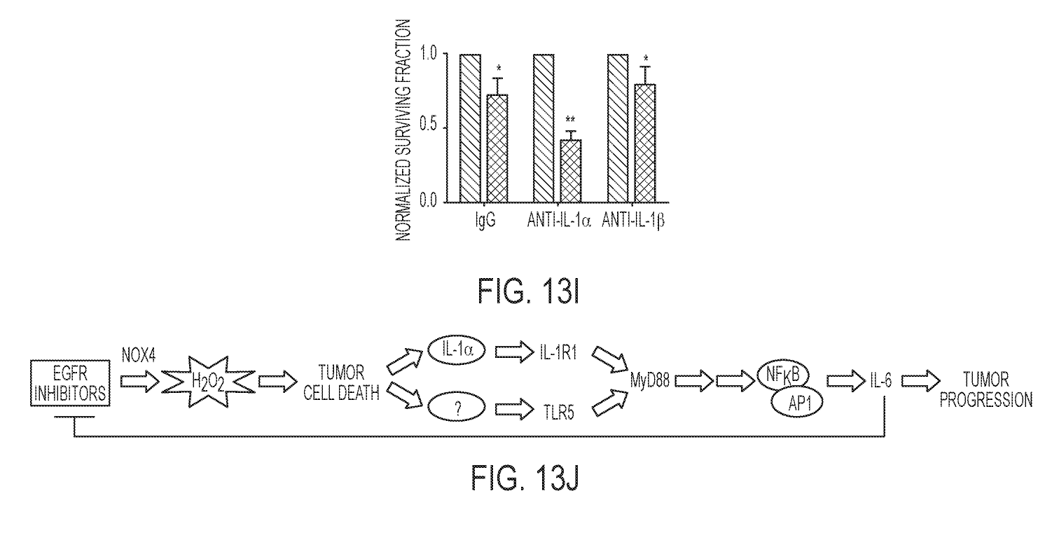

Sequenced HNSCC tumors (TCGA, n=467) with high expression of MyD88, TLRs, IL-1R, IL-18R, IL-1α, IL-1β and IL-1RA were plotted for survival against low expressing tumors (FIG. 13A-H). MyD88, TLRs, IL-18R, IL-1β and IL-1RA were not significantly correlated with survival (FIG. 13A-C, G,H). High IL-1R expressing tumors showed a trend (p=0.06) toward a negative correlation with survival (FIG. 13D) while IL-1α mRNA expression was negatively correlated (p=0.04) with survival (FIG. 13E). Selected tumors from patients that received targeted molecular therapy (TMT, n=40), showed an increased negative correlation with survival (p=0.02, FIG. 13F) suggesting that IL-1α expression may be an important prognostic marker in HNSCC.

Our results and previous findings suggest that ERL (and perhaps other EGFRIs) induce cell death via H₂O₂-mediated oxidative stress due to NOX4 activity leading to IL-1α release and activation of the IL-1R/MyD88/NFkB signaling axis on surviving tumor cells resulting in IL-6 secretion (FIG. 13J). Our results also propose that another unidentified DAMP may be released that activates the TLRS/MyD88/NFkB signaling axis resulting in IL-6 secretion. This IL-6 signaling is believed to reduce the anti-tumor activity of EGFRIs and promote tumor progression (FIG. 13J).

Discussion

Our lab has previously shown that EGFRIs increased IL-6 secretion and that IL-6 levels played a critical role in the anti-tumor effect of ERL in vitro and in vivo (10) which has been supported and studied in depth by other groups (15-18). The studies presented here now indicate that MyD88-dependent IL-1R signaling is most likely responsible for the IL-6 production induced by EGFRIs. Therefore targeting IL-1 signaling may be a novel strategy to increase the anti-tumor efficacy of ERL and other EGFRIs in HNSCC.

We have observed that the majority of cellular processes and pathways upregulated by ERL treatment were related to immune response and inflammation (FIG. 7,8). These observations support one other study showing that the EGFRI PD153035 upregulated genes related to inflammation and innate immunity (25). Interestingly, the inflammatory profile displayed by ERL treatment was remarkably similar to that of rheumatic diseases and other systemic inflammatory disorders (FIG. 7C,D). In fact, inhibition of the IL-1 pathway is a well-documented strategy for the treatment of rheumatoid arthritis (RA) since IL-1R ligands (IL-1α and IL-1β) are particularly abundant in the synovial lining of the joint (26). Anakinra is a humanized recombinant IL-1R antagonist (IL-1RA) that is FDA approved for use in the treatment of RA. IL-1RA is an IL-1R ligand that inhibits the IL-1 pathway through competition with the other IL-1R ligands (27). In support of this, we have shown that anakinra effectively blocked ERL-induced IL-6 in HNSCC cell lines (FIG. 11A,B) implying that IL-1 pathway-targeting drugs used for the management of RA (and other systemic inflammatory disorders) could be investigated as a potential adjuvant to EGFRIs in the treatment of HNSCC.

Of the ligands in the IL-1 family, IL-1β is the most well-studied and its production is dependent on inflammasome-mediated caspase-1 activity (28). In the present studies we believe that IL-1α and not IL-1β is involved in the activation of the IL-1R/MyD88/IL-6 pathway by ERL since we were unable to detect any secreted IL-1β and suppression of IL-1β using a neutralizing IL-1β antibody or a caspase-1 inhibitor did not affect ERL-induced IL-6 (FIG. 10E,G; FIG. 12A). On the other hand, we were able to detect IL-1α (FIG. 11E) and suppression of IL-1α significantly blocked ERL-induced IL-6 (FIG. 11G) suggesting that IL-1α was the ligand responsible for activating the IL-1 pathway.

Unlike IL-1β, IL-1α is not secreted from the cell, but is released during cell death and acts as a DAMP (29). It is likely that the cell death induced by ERL treatment resulted in IL-1α release since the use of ZVAD blocked ERL-induced cell death and IL-1α release (FIG. 12A). Furthermore, our laboratory has previously shown that ERL induces cell death via H₂O₂-mediated oxidative stress due to NOX4 activity (23). We have now extended these findings to show that IL-1α release in addition to downstream IL-6 secretion is mediated by ERL-induced cell death due to NOX4-induced oxidative stress (FIG. 12B-F).

Our gene expression analyses also implicated TLR/MyD88 signaling (especially TLR2) as a possible mediator ERL-induced IL-6 (FIG. 8) however we found no evidence of TLR2 involvement despite TLR2 being present and active on HNSCC tumors and cell lines (FIG. 10A-C). Surprisingly, we found that TLR2 knockdown increased IL-6 secretion (FIG. 10E). An explanation for these results is unclear although one prior report has shown that activation of TLR2 resulted in decreased NFkB activity via increased miR-329 leading to decreased IL-6 expression in human trophoblast cells (30). Perhaps in our HNSCC cell model, inhibition of TLR2 expression decreased levels of miR-329 resulting in increased NFkB and IL-6 secretion, which would be consistent with the previous findings in trophoblast cells (30). Interestingly, TLR5 was active in only SQ20B cells (FIG. 10C) and TLR5 knockdown partially but significantly suppressed ERL-induced IL-6 production in this cell line only suggesting that TLR5 activity may be important in select HNSCC cell lines (FIG. 10G,H). At this time, endogenous DAMPS capable of activation of TLR5 are unknown, therefore we are unclear as to how ERL induces TLR5.

Given that IL-1α appears to be the ligand that triggers the IL-1R/MyD88/IL-6 cascade that we believe is responsible for poor response to EGFRIs, then in theory, neutralization of IL-1α should increase the anti-tumor efficacy of EGFRIs in the same manner as blockade of IL-6 as previously shown by our laboratory (10, 15-18). The observed effects of ERL in our studies are believed to be directly due to cell death mediated by EGFR inhibition and not due to off-target effects of the drugs since 1: we are using clinical achievable doses (31) and 2: we have already confirmed the ability of EGFR knockdown (using siRNA targeted to EGFR) to induce oxidative stress, cell death and cytokine secretion (10, 23).

To further stress the importance of IL-1 a in the management of HNSCC, we found that HNSCC tumors expressed high levels of IL-1α compared to matched normal tissue (FIG. 11D) and high-IL-1α-expressing tumors have worse prognosis than low-IL-1α-expressing tumors (FIGS. 13E). Furthermore, when we selected for tumors from patients receiving TMT, we found an increased separation and significance between the survival curves (FIG. 13F) suggesting that IL-1α expression may not only predict overall survival in HNSCC but also predict response to TMT. Unfortunately, the clinical information associated with the tumors from patients that received TMT did not reveal what treatment regimen was administered therefore we cannot make firm conclusions from this analysis. However since the only TMT currently used in HNSCC is EGFR-targeting drugs and the only approved EGFRI for HNSCC to date is CTX, it is more likely than not that the TMT involved CTX in our analysis.

Suppression of MyD88 effectively blocked ERL-induced IL-6 production and suppressed tumor growth in the presence of ERL (FIG. 9), which is likely due to the ability of MyD88 knockdown to block all potential pro-inflammatory signaling from MyD88-dependent receptors. It is unclear why control-treated shMyD88 #9 tumors displayed such a pronounced inhibition of tumor growth (FIG. 9E) compared to control-treated shMyD88 #2 tumors (FIG. 9D). Previous reports have shown that MyD88 signaling may induce EGFR ligands such as amphiregulin (AREG) and epiregulin (EREG) resulting in the activation of EGFR (32). Perhaps knockdown of MyD88 expression in the shMyD88 #9 clone led to the inhibition of EGFR via downregulation of AREG/EREG in addition to suppression of IL-6, which may explain our observations. Nevertheless, these results suggest that MyD88 inhibition may also be a promising strategy to increase the effect of ERL.

It should be noted that global inhibition of MyD88, IL-1α or any factor in the IL-1R/MyD88/IL-6 signaling axis in vivo may have unexpected results. Our model takes into account only the activity of MyD88 or IL-1α within cancer cells. Inhibition of these inflammatory components in innate immune cells may change the inflammatory microenvironment especially in an immune competent mouse model, conceivably altering recruitment of immune cells and unpredictably altering growth of the tumor. This remains to be studied.

Based on these findings and our prior studies (10, 21, 23), we propose a model in which EGFR inhibition causes cell death and release of IL-1α which we believe binds its receptor IL-1R on surviving cells, activates MyD88 and induces IL-6 secretion via NFkB (FIG. 13J). IL-6 signaling pathways typically lead to phosphorylation of STAT3, which is well known to compensating for the loss of EGFR signaling due to cross talk (33). As such, we believe that the poor response and possibly acquired resistance to ERL in the clinical setting may be due to IL-1R/MyD88/IL-6 signaling triggered by release of IL-1α from dying cells, which is different from other proposed mechanisms of poor response/acquired resistance (acquired mutations, alternative signaling pathways (6-9)). To our knowledge, the studies presented here are the first to connect IL-1α and MyD88-dependent signaling with response to EGFR-targeted therapy and this novel mechanism may offer insight into why other methods of overcoming EGFRI resistance have failed, and proposes new clinical targets that may enhance the efficacy of EGFRIs in HNSCC.

### EXAMPLE 2

### References

1. Loeffler-Ragg J, Schwentner I, Sprinzl G M, Zwierzina H. EGFR inhibition as a therapy for head and neck squamous cell carcinoma. Expert Opin Investig Drugs 2008;17:1517-31.
2. Bonner J A, Harari P M, Giralt J, Azarnia N, Shin D M, Cohen R B, et al. Radiotherapy plus cetuximab for squamous-cell carcinoma of the head and neck. N Engl J Med 2006;354:567-78.
3. Cohen E E, Kane M A, List M A, Brockstein B E, Mehrotra B, Huo D, et al. Phase II trial of gefitinib 250 mg daily in patients with recurrent and/or metastatic squamous cell carcinoma of the head and neck. Clin Cancer Res 2005;11:8418-24.
4. Soulieres D, Senzer N N, Vokes E E, Hidalgo M, Agarwala S S, Siu L L. Multicenter phase II study of erlotinib, an oral epidermal growth factor receptor tyrosine kinase inhibitor, in patients with recurrent or metastatic squamous cell cancer of the head and neck. J Clin Oncol 2004;22:77-85.
5. Vermorken J B, Trigo J, Hitt R, Koralewski P, Diaz-Rubio E, Rolland F, et al. Open-label, uncontrolled, multicenter phase II study to evaluate the efficacy and toxicity of cetuximab as a single agent in patients with recurrent and/or metastatic squamous cell carcinoma of the head and neck who failed to respond to platinum-based therapy. J Clin Oncol 2007;25:2171-7.
6. Argiris A, Kotsakis A P, Hoang T, Worden F P, Savvides P, Gibson M K, et al. Cetuximab and bevacizumab: preclinical data and phase II trial in recurrent or metastatic squamous cell carcinoma of the head and neck. Ann Oncol 2013;24:220-5.
7. de Souza J A, Davis D W, Zhang Y, Khattri A, Seiwert T Y, Aktolga S, et al. A phase II study of lapatinib in recurrent/metastatic squamous cell carcinoma of the head and neck. Clin Cancer Res 2012;18:2336-43.
8. Miller V A, Hirsh V, Cadranel J, Chen Y M, Park K, Kim S W, et al. Afatinib versus placebo for patients with advanced, metastatic non-small-cell lung cancer after failure of erlotinib, gefitinib, or both, and one or two lines of chemotherapy (LUX-Lung 1): a phase 2b/3 randomised trial. Lancet Oncol 2012;13:528-38.
9. Ross H J, Blumenschein G R, Jr., Aisner J, Damjanov N, Dowlati A, Garst J, et al. Randomized phase II multicenter trial of two schedules of lapatinib as first- or second-line monotherapy in patients with advanced or metastatic non-small cell lung cancer. Clin Cancer Res 2010;16:1938-49.
10. Fletcher E V, Love-Homan L, Sobhakumari A, Feddersen C R, Koch A T, Goel A, et al. EGFR inhibition induces proinflammatory cytokines via NOX4 in HNSCC. Mol Cancer Res 2013;11:1574-84.
11. Duffy S A, Taylor J M, Terrell J E, Islam M, Li Y, Fowler K E, et al. Interleukin-6 predicts recurrence and survival among head and neck cancer patients. Cancer 2008;113:750-7.
12. Heimdal J H, Kross K, Klementsen B, Olofsson J, Aarstad H J. Stimulated monocyte IL-6 secretion predicts survival of patients with head and neck squamous cell carcinoma. BMC Cancer 2008;8:34.
13. Kamimura D, Ishihara K, Hirano T. IL-6 signal transduction and its physiological roles: the signal orchestration model. Rev Physiol Biochem Pharmacol 2003;149:1-38.
14. Riedel F, Zaiss I, Herzog D, Gotte K, Naim R, Hormann K. Serum levels of interleukin-6 in patients with primary head and neck squamous cell carcinoma. Anticancer Res 2005;25:2761-5.
15. Giles K M, Kalinowski F C, Candy P A, Epis M R, Zhang P M, Redfern A D, et al. Ax1 mediates acquired resistance of head and neck cancer cells to the epidermal growth factor receptor inhibitor erlotinib. Mol Cancer Ther 2013;12:2541-58.
16. Lee HJ, Zhuang G, Cao Y, Du P, Kim HJ, Settleman J. Drug resistance via feedback activation of Stat3 in oncogene-addicted cancer cells. Cancer Cell 2014;26:207-21.
17. Li L, Han R, Xiao H, Lin C, Wang Y, Liu H, et al. Metformin sensitizes EGFR-TKI-resistant human lung cancer cells in vitro and in vivo through inhibition of IL-6 signaling and EMT reversal. Clin Cancer Res 2014;20:2714-26.
18. Yao Z, Fenoglio S, Gao DC, Camiolo M, Stiles B, Lindsted T, et al. TGF-beta IL-6 axis mediates selective and adaptive mechanisms of resistance to molecular targeted therapy in lung cancer. Proc Natl Acad Sci USA 2010;107:15535-40.
19. Martin M U, Wesche H. Summary and comparison of the signaling mechanisms of the Toll/interleukin-1 receptor family. Biochim Biophys Acta 2002;1592:265-80.
20. Weichselbaum R R, Dahlberg W, Beckett M, Karrison T, Miller D, Clark J, et al. Radiation-resistant and repair-proficient human tumor cells may be associated with radiotherapy failure in head- and neck-cancer patients. Proc Natl Acad Sci USA 1986;83:2684-8.
21. Sobhakumari A, Schickling B M, Love-Homan L, Raeburn A, Fletcher E V, Case A J, et al. NOX4 mediates cytoprotective autophagy induced by the EGFR inhibitor erlotinib in head and neck cancer cells. Toxicol Appl Pharmacol 2013;272:736-45.
22. Spitz D R, Malcolm R R, Roberts R J. Cytotoxicity and metabolism of 4-hydroxy-2-nonenal and 2-nonenal in H2O2-resistant cell lines. Do aldehydic by-products of lipid peroxidation contribute to oxidative stress? Biochem J 1990;267:453-9.
23. Orcutt K P, Parsons A D, Sibenaller Z A, Scarbrough P M, Zhu Y, Sobhakumari A, et al. Erlotinib-mediated inhibition of EGFR signaling induces metabolic oxidative stress through NOX4. Cancer Res 2011;71:3932-40.
24. Hong D S, Hui D, Bruera E, Janku F, Naing A, Falchook G S, et al. MABp1, a first-in-class true human antibody targeting interleukin-1 alpha in refractory cancers: an open-label, phase 1 dose-escalation and expansion study. The Lancet Oncology 2014;15:656-66.
25. Woodworth C D, Michael E, Marker D, Allen S, Smith L, Nees M. Inhibition of the epidermal growth factor receptor increases expression of genes that stimulate inflammation, apoptosis, and cell attachment. Mol Cancer Ther 2005;4:650-8.
26. Burmester G R, Feist E, Dorner T. Emerging cell and cytokine targets in rheumatoid arthritis. Nat Rev Rheumato12014;10:77-88.
27. Boraschi D, Tagliabue A. The interleukin-1 receptor family. Semin Immunol 2013;25:394-407.
28. Bergsbaken T, Fink S L, Cookson B T. Pyroptosis: host cell death and inflammation. Nat Rev Microbiol 2009;7:99-109.
29. Kim B, Lee Y, Kim E, Kwak A, Ryoo S, Bae S H, et al. The Interleukin-lalpha Precursor is Biologically Active and is Likely a Key Alarmin in the IL-1 Family of Cytokines. Front Immunol 2013;4:391.
30. Garg M, Potter J A, Abrahams V M. Identification of microRNAs that regulate TLR2-mediated trophoblast apoptosis and inhibition of IL-6 mRNA. PLoS One 2013;8:e77249.
31. Hidalgo M, Siu L L, Nemunaitis J, Rizzo J, Hammond L A, Takimoto C, et al. Phase I and pharmacologic study of OSI-774, an epidermal growth factor receptor tyrosine kinase inhibitor, in patients with advanced solid malignancies. J Clin Oncol 2001;19:3267-79.
32. Brandl K, Sun L, Neppl C, Siggs O M, Le Gall S M, Tomisato W, et al. MyD88 signaling in nonhematopoietic cells protects mice against induced colitis by regulating specific EGF receptor ligands. Proc Natl Acad Sci U S A 2010;107:19967-72.
33. Colomiere M, Ward A C, Riley C, Trenerry M K, Cameron-Smith D, Findlay J, et al. Cross talk of signals between EGFR and IL-6R through JAK2/STAT3 mediate epithelial-mesenchymal transition in ovarian carcinomas. Br J Cancer 2009;100:134-44.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of treating cancer in a subject, the method comprising administering to the subject a combination therapy comprising administering (1) at least one anti-cancer therapy, and (2) a pharmaceutical composition comprising a pharmaceutically acceptable carrier and an amount of an IL-1α inhibitor, wherein the combination therapy is effective to reduce at least one symptom of the cancer in the subject.

2. The method of claim 1, wherein the at least one anti-cancer therapy is radiation therapy.

3. The method of claim 1, wherein the at least one anti-cancer therapy is a chemotherapeutic agent.

4. The method of claim 3, wherein the chemotherapeutic agent is an epidermal growth factor receptor (EGFR) inhibitor.

5. The method of claim 4, wherein the EGFR inhibitor is Erlotinib, lapatinib, cetuximab or panitumumab.

6. The method of claim 4, wherein the EGFR inhibitor is Erlotinib.

7. The method of claim 3, wherein the chemotherapeutic agent is Methotrexate (Abitrexate, Folex, Methotrexate LPF, Mexate, or Mexate-AQ), Fluorouracil (Adrucil, Fluoroplex, or Efudex), Bleomycin (Blenoxane), Cisplatin (Platinol, Platinol-AQ), Docetaxel (Taxotere), carboplatin (Paraplatin, Paraplatin-AQ) and/or paclitaxel (Abraxane, Taxol).

8. The method of claim 1, wherein the at least one anti-cancer therapy is immunotherapy.

9. The method of any one of claims 1 to 8, wherein the IL-1α inhibitor is an anti-IL-la monoclonal antibody (mAb), or a fragment thereof.

10. The method of claim 9, wherein the mAb comprises a complementarity determining region of an anti-IL-1α mAb.

11. The method of any one of claims 1 to 8, wherein the IL-1α inhibitor is anakinra.

12. The method of any one of claims 1 to 11, wherein the cancer is a hematopoietic cancer.

13. The method of any one of claims 1 to 11, wherein the cancer is a solid tumor.

14. The method of claim 13, wherein the cancer is a HNSCC tumor.

15. The method of claim 13 or 14, wherein the tumor is decreased in size in the subject by at least about 10%.

16. The method of any one of claims 1 to 15, wherein the composition and/or the anti-cancer therapy is administered parenterally.

17. The method of any one of claims 1 to 15, wherein the composition and/or the anti-cancer therapy is administered intramuscularly, subcutaneously, intradermally or intravenously.

18. The method of any one of claims 1 to 15, wherein the composition and/or the anti-cancer therapy is administered orally or intranasally.

19. The method of any one of claims 1 to 18, wherein the composition and the anti-cancer therapy are administered simultaneously.

20. The method of any one of claims 1 to 18, wherein the composition and the anti-cancer therapy are administered sequentially.

21. The method of claim 20, wherein the composition is administered prior to the administration of the anti-cancer therapy.

22. The method of claim 21, wherein administration of the composition begins about 1 to about 10 days before administration of the anti-cancer therapy.

23. The method of claim 20, wherein administration of the anti-cancer therapy begins about 1 to about 10 days before administration of the composition.

24. The method of any one of claims 20 to 23, wherein administration of the composition and the anti-cancer therapy begin on the same day.

25. The method of any one of claims 1 to 24, wherein the subject is a mammal.

26. The method of claim 25, wherein the mammal is a human.

27. A pharmaceutical composition for treating cancer in a subject comprising a pharmaceutically acceptable carrier, an amount of chemotherapeutic agent, and an amount of an IL-1α inhibitor, wherein the composition is effective to reduce at least one symptom of the cancer in the subject.

28. The pharmaceutical composition of claim 27, wherein the chemotherapeutic agent is an epidermal growth factor receptor (EGFR) inhibitor.

29. The pharmaceutical composition of claim 28, wherein the EGFR inhibitor is Erlotinib, lapatinib, cetuximab or panitumumab.

30. The pharmaceutical composition of claim 29, wherein the EGFR inhibitor is Erlotinib.

31. The pharmaceutical composition of claim 27, wherein the chemotherapeutic agent is Methotrexate (Abitrexate, Folex, Methotrexate LPF, Mexate, or Mexate-AQ), Fluorouracil (Adrucil, Fluoroplex, or Efudex), Bleomycin (Blenoxane), Cisplatin (Platinol, Platinol-AQ), Docetaxel (Taxotere), carboplatin (Paraplatin, Paraplatin-AQ) and/or paclitaxel (Abraxane, Taxol).

32. The pharmaceutical composition of any one of claims 27 to 31, wherein the IL-1α inhibitor is an anti-IL-1α monoclonal antibody (mAb), or a fragment thereof.

33. The pharmaceutical composition of claim 32, wherein the mAb comprises a complementarity determining region of an anti-IL-1α mAb.

34. The pharmaceutical composition of any one of claims 27 to 34, wherein the IL-la inhibitor is anakinra.

35. The pharmaceutical composition of any one of claims 27 to 34, wherein the cancer is a hematopoietic cancer.

36. The pharmaceutical composition of any one of claims 27 to 34, wherein the cancer is a solid tumor.

37. The pharmaceutical composition of claim 36, wherein the cancer is a HNSCC tumor.

38. A kit comprising at least one anti-cancer therapy and a pharmaceutical composition comprising a pharmaceutically acceptable carrier and an amount of an IL-1α inhibitor, a container, and a package insert or label indicating the administration of the anti-cancer therapy and the IL-1α inhibitor, for reducing at least one symptom of the cancer in the subject.
